(19) United States
(12) Patent Application Publication
(10) Pub. No.: US 2009/0291883 A1
(43) Pub. Date: Nov. 26, 2009

(54) COMBINATION OF AN IMMUNOSUPPRESSIVE AGENT AND NONSTEROIDAL ANTI -INFLAMMATORY DRUGS TO TREAT DISEASE

(75) Inventors: M. Michael Wolfe, Newton Centre, MA (US); Min Yao, Brighton, MA (US)

Correspondence Address: RONALD I. EISENSTEIN, 100 SUMMER STREET, NIXON PEABODY LLP, BOSTON, MA, 02110, US

(73) Assignee: BOSTON MEDICAL CENTER CORPORATION, Boston, MA (US)

(21) Appl. No.: 12/373,841

(22) PCT Filed: Jul. 16, 2007
(86) PCT No.: PCT/US07/16102

§ 371 Date: Jan. 14, 2009

Related U.S. Application Data
(60) Provisional application No. 60/830,782, filed on Jul. 14, 2006.

(51) Int. Cl. A61K 38/13; A61K 31/519; A61K 31/315; A61K 31/341

(52) U.S. Cl. 514/11; 514/255.05; 514/274; 514/473

(57) ABSTRACT

The present invention provides methods and compositions for the treatment and prevention of neoplasia by administering an effective amount of a NSAID in combination with an effective amount of an immunosuppressant agent. In particular, the present invention provides methods and compositions for the treatment and prevention of neoplasia by administering an effective amount of COX-2 inhibitor in combination with an effective amount of an immunosuppressant agent.

### CROSS REFERENCE TO RELATED APPLICATIONS

This application claims priority under 35 U.S.C. 119(e) of U.S. Provisional Patent Application Ser. No. 60/830,782 filed 14 Jul., 2006, Express Mail No: EV 679164044 the contents of which are incorporated herein by reference in their entirety.

### BACKGROUND OF THE INVENTION

1. Technical Field

The present invention relates to the use of a combination therapy comprising an NSAID and an immunosuppressive agent. More specifically, the present invention relates to the use of the combination therapy for treating cancer and treating and/or preventing secondary malignancies in organ transplant subjects.

2. Background Art

Colorectal polyps are extremely common in adults in Western countries, they are found in more than 30% of autopsies conducted on people greater than 60 years of age (Correa P., Gastroenterology 77:1245-1251 (1979)). The colonic polyp has been implicated as a precursor in the development of colorectal cancer (Morson B. C., Cancer 34:845-850 (1974)). Current data suggest a polyp to cancer sequence, with colorectal neoplasmic changes as a continuous process from normal mucosa, to adenoma, and then to carcinoma (Schottenfeld, D. & Winawer, S. J., Cancer: Epidemiology and Prevention, Philadelphia, W.B. Saunders, 703-727 (1982)).

Histological, polyps are classified as neoplastic, i.e., adenomas, with malignant potential or as non-neoplastic, known as benign adenomas (Fenoglio, C. M. & Pascal, R. R., Cancer 50:2601-2608 (1982)). Approximately 70% of polyps removed at colonoscopy are adenomas, (Konishi, F. & Morsor, B. C., J. Clin. Pathol. 35:830-841 (1982)) with the potential to become larger than 10 mm, and therefore, having the probability of becoming tumorigenic. It is, therefore, of great importance to identify colonic polyps and to treat them before they can become malignant.

Most commonly, polyps are described as sporadic, arising spontaneously in about a quarter of the population by age 50, with the prevalence increasing with age, and which may or may not result in colorectal cancer (Winawer, S. J., et al., Gastroenterology 112:594-642 (1997)).

Familial adenomatous polyposis (FAP), on the other hand, is an autosomal dominant, inherited disorder, characterized by the presence of hundreds of adenomatous polyps in young adults and in the eventual development of colorectal cancer (Schussheim, A., et al., Gastroenterol. Nutr. 17:445-448 (1993)).

Colonoscopy is considered the best method for detecting polyps accurately, especially those measuring less than 10 mm in diameter (Rex, D. K., et al., Gastroenterology 112:17-23 (1997)). Most polyps found during colonoscopy can be completely and safely removed by electrocautery (fulguration) (Knutson, C. D. & Max, M. H., Arch Surg 114:30-435 (1979)). Some complications, however, may develop during colonoscopy, most commonly perforation and bleeding, occurring in 0.1 to 0.2% of subjects (Rankin, G. B., Gastrointestinal Endoscopy, Philadelphia, w. B. Saunders, 875-878 (1987)). In addition, it is not always possible to detect all polyps using colonoscopy because of the anatomy of the colon. In fact, in a recent study, it was shown that a carefully performed complete colonoscopy by an experienced examiner will miss an average of about 24% of polyps that are less than 10 mm in diameter (Rex, D. K., et al., Gastroenterology 1 12:24.28 (1997)).

### Non-Steroidal Anti-Inflammatory Drugs and Colorectal Polyps

To overcome the current technical limitations of colonoscopy, and to avoid the need for surgical procedures, extensive research has been focused during the past decade on finding pharmacologic agents that might be used to treat or prevent colorectal polyps. Especially, the effect of non-steroidal inflammatory drugs (NSAIDs) on colorectal polyps has become of interest.

Epidemiological studies have reported that chronic aspirin use in a study has been associated with a 50-70 percent reduction in the incidence of colorectal cancer (Logan, R. F. A., et al, Br. Med. J. 307:285-289 (1993); Rosenberg, L., et al., J. Natl. Cancer Inst. 83:355-358 (1991); Thun, M. J., et al., New Engl. J. Med. 325:1593-1596 (1991); Suh, O., et al., Cancer 72:1171-1177 (1993); Peleg II et al., Arch. Intern. Med. 65 154:394-399 (1994)). In addition, multiple animal studies have documented a chemoprotective effect of selected NSAIDS as judged by a reduction in the frequency and number of premalignant and malignant lesions (Reddy, B. S., et al, Cancer Res. 50:2562˜2568 (1990); Reddy, B. S., et al., Carcinogenesis 14:1493-1497 (1993); Rao, C. V., et al., Cancer Res. 51:4528-4534 (1991); Craven, P. A., & DeRuberis, F. R., Carcinogenesis 14:541-546 (1992); Northway, M. G., et al., Cancer 66:2300-2305 (1990); Moorghen, M., et al., J. Pathol 56:341-347 (1988); Reddy, B. S., et al, Cancer Res. 47:5340-5346 (1987); Reddy, B. S., et al., Carcinogenesis 13:1019-1023 (1992); Skinner, S. A., et al, Arch. Surg. 126:1094-1096 (1991)). In a recent case study of a subject with villous adenomas of the cecum, who refused surgical resection, a course of NSAID therapy using piroxicam, 30 mg weekly, showed dramatic and sustained regression of the pre-malignant adenomas for up to 20 months (Gowen, G. F., Dis. Colon Rectum 39:101-102 (1996)). In clinical studies of familial adenomatous polyposis, using the NSAID sulindac, at a daily dose of 300 mg, taken systemically, it was shown that the number and size of colonic polyps was significantly decreased (Giardelio, F. M., et al., New Engl. J. Med. 328:1313.1316 (1993); Labaylle, D., et al., Gastroenterology 101:635-639 (1991); Waddell, W. R., et al., Am. J. Surg. 157:175-179 (1969)). In a small pilot study, in which sulindac or piroxicam was used against sporadic colonic polyps, however, there was no similar regression of adenomatous polyps (Ladenheim, J., et al., Gastroenterology 108:1083-1087 (1995); Hixson, L. J. I et al., Am. J. Gastroenterol 88:1652-1656 (1993)). These results, however, were disputed in a more recent multicenter study of nearly 100 subjects, with sporadic polyps of 4.12 mm. When sulindac, 300 mg daily, or sulindac, 150 mg daily, or placebo, were given for one year, it was demonstrated that sulindac regardless of dose, induced regressions and prevented the progression of sporadic colorectal adenomas (DiSano, J. A., et al., Gastroenterology 112 (Suppl):555A (1997)).

### NSAIDs and Apoptosis

The precise mechanism responsible for the anti-neoplastic effect of NSAIDs is unknown. A number of recent publications have suggested that NSAIDs can be accomplishing these chemoprotective effects by induction of apoptosis, the “programmed cell death” phenomenon (Savill, J., Eur. J. Clin. Invest. 24:715-723 (1994); Thompson, C. B., Science 267:1456-1462 (1995); Bright, J. and Khar, A., Biosci Rep. 14:67-81 (1994)). In 1965. Lockshin and colleagues discussed the concept of “programmed cell death” to describe the phenomenon that had long been observed in embryogenesis where certain predetermined cells in the embryo would die at a particular stage during development (Lockshin, R. A. and Williams, C. M. J. Insect Physiol. 11:123-133 (1965)). In 1972, Kerr et al, linked this concept with a mode of cell death, defined on strict morphological criteria such as the detachment of a cell from its substratum, coupled by the fragmentation of the nucleus and cytoplasm, in a process which, they termed “apoptosis.” (Kerr, J. F. R., et al., Br. J Cancer 26:239-257 (1972)). This active cell death, under tight genetic control, is found in all tissues, and is responsible both for regulating cell number and type, as well as for disposing cells with damaged or mutant DNA. Defects in apoptosis, however, can lead to cancer, autoimmune disease and neurodegeneration (Pritchard, D. and Watson, A. J. M., Pharmacol Ther. 72:149-169 (1996)).

Defective apoptosis has been implicated in the pathogenesis of colorectal cancer. In 1995, Bedi et al. quantified the amount of apoptosis in frozen sections of 115 biopsies of colorectal epithelium from normal mucosa, adenomas from subjects with familial adenomatous polyposis, sporadic adenomas, and carcinomas by in situ nick end labeling of histopathological specimens cultured for up to 24 hours on plastic. There was progressive inhibition of apoptosis during the transformation of normal epithelium into carcinomas (Bedi, A., et al., Cancer Res. 55:1811-1816 (1995)).

Additionally, other studies support the contention that NSAIDs may exert their effect on colorectal polyps and carcinoma by inducing apoptosis. Pasricha et al. investigated the rate of proliferation and apoptosis in the flat colorectal mucosa of subjects with familial adenomatous polyposis after treatment with sulindac. No effects on proliferation were reported, but the sulindac-treated group was reported to have increased levels of colonic mucosal apoptosis (Pasncha, P. J., et al, Gastroenterology, 109:994-999 (1995)). Piazza et al. similarly reported the induction of apoptosis in an HT-29 colon adenocarcinoma cell line following sulindac administration, but reported no evidence of cell proliferation or differentiation (Piazza, R., et al., Cancer Res. 55:31 10-3116 (1995)). In a clinical study, Lee reported that there were increased levels of apoptotic bodies in colonic biopsies from subjects with diclofenac-induced colitis (Lee, F. D., J Gun Pathol 46:18-122 (1993)).

### Induction of Apoptosis via COX-2

NSAIDs are known potent inhibitors of cyclooxygenase (COX) enzymes, resulting in decreased prostaglandin synthesis, which, in turn, induces tumor cell apoptosis. Other studies have reported that NSAIDs also promote apoptosis through COX-independent pathways. A combination of these effects can be a source of the anti-neoplastic effect of NSAIDs, which continues to be an area of intense research.

The mechanism of inhibition of COX activity by aspirin and NSAIDs was first described by Vane in 1971. In the early 1990s, several groups reported the discovery of two COX isoforms. COX-1, the constitutively expressed form, appears to function as a physiological regulatory enzyme in most tissues. COX-2, however, is strongly inducible and plays an integral role in several physiological and pathological processes, including cell proliferation and inflammatory responses. COX-2 mRNA expression and protein were reported to be enhanced in human colorectal adenomas and adenocarcinomas. Conversely, specific COX-2 inhibition, either by targeted knockout of the COX-2 gene or by pharmacological intervention, has been reported to effectively decrease the growth of murine intestinal adenomas. In a rat model of chemically-induced colorectal cancer, the COX-2 selective inhibitor celecoxib suppressed the formation of aberrant crypt foci, precursors of adenomas. Celecoxib was also reported to have inhibited the incidence and multiplicity of colon tumors by 93% and 97%, respectively.

As noted above COX-2 overexpression is important during colorectal carcinogenesis; however it is unclear exactly where in the multistep process COX-2 deregulation occurs. Because COX-2 overexpression occurs even in small adenomas, it is thought to represent an early event, promoting tumor proliferation and suppression of apoptosis. In vitro, many NSAIDs including sulindac, indomethacin, naproxen, peroxicam, aspirin, and COX-2 specific inhibitors are reported to cause apoptosis in colon cancer cells. Thus, a growing body of experimental data supports the hypothesis that NSAIDs exert their chemopreventive effect by restoring to normal the frequency of apoptosis in colonic mucosa.

Hereditary colorectal cancer (CRC), best defined in the familial adenomatous polyposis (FAP) and hereditary nonpolyposis colorectal cancer (HNPCC) syndromes, has been estimated to account for 15% of all colon cancers occurring in the general population. Chemoprevention is of special interest in subjects with germline mutations resulting in FAP or HNPCC since these subjects are at risk for the development of tumors in multiple organs. Most of the studies to date have concentrated on the role on NSAIDs in FAP.

In brief, FAP results from mutations in the APC gene (chromosome 5q), and pedigree analysis reveals an autosomal dominant pattern with nearly complete penetrance. Typically, subjects with FAP develop diffuse colorectal polyposis by their adolescent years, and, if untreated, will be diagnosed with colorectal cancer by 40 years of age. Currently, the most effective management is initial intensive endoscopic surveillance starting at age 10-12. Once the number of polyps increases to a level that eliminates the possibility of safe endoscopic treatment, prophylactic total colectomy or subtotal colectomy followed by annual endoscopy of the remaining rectum is recommended. Subjects with FAP are also at an increased risk for other malignancies, particularly small bowel, stomach, and extraintestinal neoplasia such as desmoid tumors, papillary thyroid carcinoma, sarcomas, hepatoblastoma, pancreatic carcinoma, and medulloblastoma. Although it is recommended that FAP subjects be screened for duodenal adenomas with upper endoscopy, screening for other extracolonic tumors is not routinely performed.

Chemoprevention in FAP has been studied intensively both in animals and humans, using agents such as ascorbic acid, calcium, and, most promisingly, NSAIDs. FAP research has benefited from a mutant mouse model with multiple intestinal neoplasias (Min), which harbors mutations of the APC gene. Multiple studies using the Min mouse model have provided encouraging results with both non-selective and selective NSAIDs, reducing tumor burden, size, and multiplicity. In humans, numerous uncontrolled and controlled studies have evaluated the effects of NSAIDS, particularly sulindac, and the more selective COX-2 inhibitor celecoxib, on polyp size and number. The data are encouraging, and celecoxib was shown in one study to cause regression of polyps in subjects with FAP However, studies have additionally shown that discontinuation of treatment results in the recurrence of polyps, and subjects on sulindac therapy have been reported to develop CRC. Therefore, due to concerns regarding the length of therapy required, the development of CRC in subjects on treatment, chemoprevention with NSAIDS has not replaced conventional endoscopic surveillance and colectomy recommendations in FAP subjects.

Of particular interest in FAP are common extracolonic malignancies such as desmoid tumors and duodenal adenocarcinomas, which represent important targets for chemoprevention agents. Studies have shown that sulindac can be effective in the treatment of early duodenal adenomas in FAP. Celecoxib at a dose of 400 mg twice daily has been shown to decrease duodenal polyposis in subjects with clinically significant disease at baseline (greater than 5% covered by polyps), a 31% reduction in involved areas compared with 8% on placebo (p=0.049). No studies have demonstrated the utility of NSAIDs in treating desmoid tumors.

Currently, no definitive recommendations have been formulated regarding the use of NSAIDs in subjects with FAP. Based upon the studies mentioned above, some centers are increasingly using NSAIDs to delay the necessity for colectomy, and thereafter to decrease polyp formation in the retained rectum after subtotal colectomy. Celecoxib was approved recently by the U.S. Food and Drug Administration for use in this setting. Additionally, the use of NSAIDs, particularly celecoxib, appears to be a useful adjunct in subjects with duodenal polyposis.

Chemoprevention with NSAIDs in HNPCC has not been as vigorously evaluated either in animal models or in human trials. Interestingly, COX-2 is overexpressed less commonly in HNPCC than in sporadic colorectal cancer. Conversely, aspirin and sulindac have been shown to reduce the microsatellite instability (MSI) phenotype of CRC cell lines caused by mutations in the hMLHI, hMSH2 and hMSH6 genes. Further studies are required, and an ongoing trial, Concerted Action Polyp Prevention (CAPP II), will shed further light on the role of NSAIDs in HNPCC.

### Local Delivery of NSAIDs to the Colon

A disadvantage of most NSAID therapy for colorectal polyps is that the NSAID is given systemically, and for long periods. Prolonged high systemic concentrations of many NSAIDs can result in other complications unrelated to the polyp treatment. For example, such NSAID users have a three-fold greater risk of developing serious gastrointestinal (GI) complications over non-NSAID users. It has been estimated that 20% to 40% of subjects on systemic NSAID therapy develop peptic ulcers (Taha, A., et al., N. Engl. J. Med. 334:1435-1439 (1996)). It has also been estimated that 10,000-20,000 fatalities a year occur in the United States from NSAID-induced gastrointestinal disorders. Other adverse effects of NSAIDs include renal failure, hepatic dysfunction, bleeding and gastric ulceration. The side effects of NSAIDs are especially of concern in the elderly, the very population most at risk for the development of colonic polyps. Therefore, a need exists for an alternative method to target therapeutic concentrations of NSAIDs to the site of colonic polyps.

Sulindac, given orally as a tablet, is primarily absorbed through the gastrointestinal tract. The peak plasma concentration is reached about two hours after dosing (Swanson, B. N. et al., Gun. Pharmacol. Ther. 32:397-403 (1982)). As a result of sulindac's extensive first-pass metabolism to its active metabolites, the plasma concentrations of sulindac sulfide and sulindac sulfone will exceed the levels of sulindac within four hours after dosing. Thereafter, these metabolites will remain the two major components in the blood while the concentration of sulindac will rapidly taper off (Duggan, D. E. et al., Clin. Pharmacol. Ther. 21:326-335 (1977)).

It has been demonstrated that although sulindac that is administered orally is primarily absorbed into the blood, a certain amount reaches the colon. The sulindac that reaches the colon will be reduced by the colonic microflora exclusively to sulindac sulfide, resulting in a high lumenal concentration of sulindac sulfide in the colon (Hanif, R. et al., Biochem. Pharmacol. 52:237-245 (1996)). The sulindac sulfide that is formed will then be absorbed through the colon walls to the bloodstream. This premise is supported by the fact that sulindac sulfide appears in the plasma long after sulindac and sulindac sulfone have been excreted in the urine and feces and that these findings are not seen in subjects who underwent a colectomy and ileostomy (Strong, H. A. et al., Clin. Pharmacol. Ther. 38:387-393 (1985)). It can be concluded that the intact colon plays a significant role in the sustained presence of sulindac sulfide in the blood and that delivering the entire dose of the sulindac to the colon will result in a significant enhancement of the formation of sulindac sulfide over the less active metabolite, the sulindac sulfone.

The most promising arena for NSAID use is secondary prophylaxis in subjects who have had CRC or aderiomas in the past. These subjects are at an increased risk for developing further lesions, and the benefit of prophylaxis may thus outweigh the risks of major gastrointestinal bleeding or hemorrhagic stroke associated with the use of these agents. Further studies need to address whether NSAID prophylaxis can reduce current endoscopic surveillance recommendations. In addition, long-term studies evaluating the use of selective COX-2 inhibitors, which appear to carry a lower risk for the development of GI hemorrhage, will be necessary before recommending their use for not only secondary, but also primary, CRC prophylaxis.

The use of NSAIDS or aspirin in primary prevention of CRC has not been validated. Chemoprophylaxis in average-risk subjects would require regular aspirin use for at least 10 to 20 years, and the cumulative risk of major adverse events may outweigh any reduction in CRC risk. Interestingly, although the benefit of aspirin in coronary artery disease has been established, the U.S. Preventive Services Task Force does not recommend aspirin unless the 5-year risk for CAD is at least three 3 percent. In fact, cost-effectiveness analyses of aspirin for primary prophylaxis have shown an increase in cost per life saved when compared to any of the current screening recommendations.

### Local Delivery of Drugs to the Colon

U.S. Pat. No. 5,498,608 (Johnson, L. K.) describes the use of 2-hydroxy-5-2 8 5 phenylazobenzoic acid derivatives for the treatment of colon cancer. The derivatives are prodrugs that are converted into an active antiinflammatory drug by the action of colonic bacteria. The use of these agents for the treatment or prevention of colon cancer is discussed.

U.S. Pat. No. 5,686,589, U.S. Pat. No. 5,401,774, U.S. Pat. No. 5,643,959, EP 485,171, EP 485,173, and EP 508,586 (Brendel, K.) describes conjugating drugs into a prodrug form with substituted fused ring phenylacetic acids as a mechanism to deliver the active agent, such as an NSAID, to a colonic polyp. Colonic bacterial enzymes then cleave the active agent from the macromolecule.

U.S. Pat. No. 5,686,105 and U.S. Pat. No. 5,686,106 (both to Kelm, G. R.) describe the use of polymers to coat an active agent for delivery to the colon. The polymers dissolve at about the time that the dosage form reaches the inlet between the small intestine and the colon, or thereafter in the colon. Examples of such polymers include Eudragit® L and cellulose acetate phthalate. Examples of the types of agents that can be provided to the colon in this manner include agents for the topical treatment of diseases of the colon, such as irritable bowel syndrome, Crohn's disease, ulcerative colitis and carcinomas. Examples of the specific active agents that are listed include nonsteroidal antiinflammatory drugs, and chemotherapeutics for treatment of carcinomas.

U.S. Pat. No. 5,464,633 (Conte, U., et al.) describes a tablet that consists of a core containing the active substance, and an external layer that is able to prevent the immediate release of the active substance. The external layer can be a natural and/or synthetic polymeric substance in the class of the erodible and/or geliable and/or soluble in an aqueous medium hydrophilic polymers and adjuvant substances. Lastly, the layer is surrounded by a gastroresistant and enterosoluble coating.

The incidence of post-transplant malignancy has been estimated at 20% after ten years of chronic immunosuppression. Post-transplant recipients are not only at greater risk for developing colorectal cancer (CRC), but at each cancer stage, these individuals have markedly lower survival rates compared with the general population. Studies in patients with CRC taking nonsteroidal anti-inflammatory drugs (NSAIDs) have shown a reduction in mortality.

The use of NSAIDS for the treatment of cancer is typically less effective than standard anti-cancer therapies such as use of chemotherapeutic agents, radiotherapy and surgical resection. Accordingly, there is still a need for improved treatments of cancer using NSAIDS. It would be useful to develop new more effective cancer treatments using NSAIDS.

### SUMMARY

The present invention relates to, in part, compositions comprising NSAIDs and immunosuppressive agents for the administration to subjects. In particular, the present invention provides methods to prevent cancer in a subject when a subject is administered an immunosuppressive agent.

Immunosuppressive therapy is necessary for the prevention of rejection of transplanted organs and is used in treatment of auto-immune diseases. However, one serious side effect of immunosuppression or immunosuppressive therapy is the development of secondary hematological and solid malignancies. The inventors have surprisingly discovered that long-term administration of a non-steroidal anti-inflammatory drug (NSAID) in combination with an immunosuppressive agent prevents such secondary tumor growth and also prevents tissue rejection. Moreover, the long-term administration of a non-steroidal anti-inflammatory drug (NSAID) in combination with an immunosuppressive agent can also be used for the treatment of existing tumors. Given the level and the number of tumors that develop as a result of immunosuppressive therapy, for example the number of solid tumors and secondary hematological tumors in subject administered an immunosuppressive agent, and the fact that NSAIDS are not typically as effective anti-cancer agents as other anti-cancer therapies such as chemotherapy or radiotherapy, it is surprising that administration of NSAIDS for example selective cyclooxygenase-2 (COX-2) inhibitors for an extended period of time can prevent tumor growth and treat existing tumors. Accordingly, the inventors have discovered a method that is a chemopreventative strategy enabling continuous administration of an immunosuppressive agent without tumor growth.

Accordingly, one aspect of the present invention provides a method of treating and/or preventing a malignancy or neoplasia disorder in a subject at risk thereof, the method comprising administering to the subject a NSAID, for example a cyclooxygenase-2 inhibitor or a pharmaceutically acceptable salt, ester or prodrug thereof in a first amount and an immunosuppressant agent, for example cyclosporine or a derivative or analogue thereof in a second amount, wherein the first amount together with the second amount comprises a therapeutically effective amount for the treatment and/or prevention of a malignancy or neoplasia disorder in the subject.

In particular, the present invention requires for a NSAID, such as COX-2 inhibitor to be administered for an extended period of time, for example for at least 1 month, for at least 2 months, for at least 3 months when the immunosuppressive agent, for example cyclosporine, is also administered to the subject. For example, in some embodiments a NSAID, such as COX-2 inhibitor to be administered for the whole period when the immunosuppressive agent is being administered to the subject. In some embodiments, the NSAID is administered at regular scheduled intervals or on a continued basis for a minimum of one month. It should be noted that the administration of the NSAID according to the methods of the present invention is different from administration of NSAIDs for other therapeutic interventions or for the treatment of other indications, such as headache or muscle ache, when NSAIDs are typically administered for short periods of time for example for 1 or 2 days or up to a week, and administration is sporadic, in other words, administration begins on presentation of symptoms and is not planned or scheduled administration.

In some embodiments, first amount is from about 0.001 to about 100 mg/day per kg of body weight of the subject and the second amount is from about 1 to about 600 mg/day per kg of body weight of the subject. In some embodiments, the first amount is from about 0.5 to about 50 mg/day per kg of body weight of the subject and the second amount is from about 100 to about 500 mg/day per kg of body weight of the subject. In alternative embodiments, the first amount is from about 1 to about 20 mg/day per kg of body weight of the subject and the second amount is from about 200 to about 400 mg/day per kg of body weight of the subject.

Another aspect of the present invention relates to a composition for the treatment, prevention or inhibition of a malignancy or neoplasia disorder in a subject with, or at risk of developing a malignancy or neoplasia disorder, the composition comprising a NSAIDS or an isomer or a pharmaceutically acceptable salt, ester or prodrug thereof in a first amount and an immunosuppressive agent in a second amount, wherein the first amount together with the second amount comprises a therapeutically effective amount for the treatment, prevention or inhibition of a malignancy, or neoplasia disorder in the subject.

In particular, the subject at risk of developing a malignancy or neoplasia disorder is a subject receiving or scheduled to receive immunosuppression. Accordingly, the methods or compositions as disclosed herein involving regular or continued administration of NSAIDS and administration of immunosuppressive agent to a subject scheduled to receive or who is receiving immunosuppression in order to prevent or minimize tumor growth and the occurrence of secondary tumors while still maintaining effective immunosuppression.

A further aspect of the present invention relates to a composition for the treatment, prevention or inhibition of a malignancy or neoplasia disorder in a subject with, or at risk of developing, a malignancy or neoplasia disorder, the composition comprising a COX-2 inhibitor or a pharmaceutically acceptable salt, ester or prodrug thereof in a first amount and cyclosporine in a second amount, wherein the first amount together with the second amount comprises a therapeutically effective amount for the treatment, prevention or inhibition of a malignancy, or neoplasia disorder in the subject.

In some embodiments, the cyclooxygenase-2 inhibitor or isomer, pharmaceutically acceptable salt, ester, or prodrug thereof and the immunosuppressive agent are administered substantially simultaneously. In some embodiments, the cyclooxygenase-2 inhibitor or isomer, pharmaceutically acceptable salt, ester, or prodrug thereof and the immunosuppressive agent are administered sequentially.

In some embodiments, the cyclooxygenase-2 inhibitor is selected from the group comprising celecoxib (B-18), valdecoxib (B-19), deracoxib (B-20), rofecoxib (B-21), etoricoxib (MK-663; B-22), JTE-522 (B-23), lumiracoxib, meloxicam etodolac or an isomer, a pharmaceutically acceptable salt, ester, or prodrug thereof, for example, one such prodrug of a cyclooxygenase-2 inhibitor is paracoxib. In some embodiments, the cyclooxygenase-2 inhibitor is rofecoxib (B-21) or an analogue, isomer, prodrug or derivative thereof.

In some embodiments, the COX-2 inhibitor or isomer, pharmaceutically acceptable salt, ester, or prodrug thereof has a COX-2 IC50 of less than about 5 μmol/L, for example a selectivity ratio of COX-2 inhibition to Cox-1 inhibition of at least about 1.5. In some embodiments, a COX-2 inhibitor or isomer, pharmaceutically acceptable salt, ester, or prodrug thereof has a COX-2 IC50 of less than about 1 μmol/L and a selectivity ratio of COX-2 inhibition to Cox-1 inhibition of at least about 100. In further embodiments, the COX-2 inhibitor or isomer, pharmaceutically acceptable salt, ester, or prodrug thereof has a Cox-1 IC50 of at least about 1 μmol/L, for example at least about 20 μmol/L.

In some embodiments, the immunosuppressive agent useful in the methods and compositions as disclosed herein is for example, but not limited, selected from the group consisting of cyclosporin, cyclosporin A, FK506, rapamycin, leflunomide, deoxyspergualin, prednisone, azathioprine, mycophenolate mofetil, OKT3, ATAG, mizoribine, mycophenolic acid, azathioprine or tacrolimus or an analog, hydrolysis product, metabolite or precursor thereof. In some embodiments the immunosuppressive agent is cyclosporin or an analog, hydrolysis product, metabolite or precursor thereof.

In some embodiments, the methods and compositions as disclosed herein are useful for administration to subject, for example an animal, and preferably where the subject is a human. In some embodiments, the subject is scheduled to receive or who is receiving immunosuppression.

In some embodiments, the subject has an increased risk of developing a malignancy, cancer or neoplasm. In alternative embodiments, the subject is in need of immunosuppressive therapy, for example a subject who is a bone marrow transplant recipient or organ transplant recipient. In some embodiments, a subject has had, is about to have or is having a bone marrow transplant and/or organ transplant.

In alternative embodiments, the subject scheduled to received or is receiving immunosuppression is a subject in need of immunosuppressive therapy has an auto-immune disease, for example the subject has, or is at increased risk of having an auto-immune disease that is selected from the group, but not limited to lupus erythematosus, Sjogren's syndrome, Hashimoto thyroiditis, rheumatoid arthritis, juvenile (type 1) diabetes, polymyositis, scleroderma, Addison disease, vitiligo, pernicious anemia, glomerulonephritis, graft versus host disease (GVH), multiple sclerosis, and pulmonary fibrosis.

In some embodiments, the methods and compositions are administered enterally or parenterally in one or more doses per day. In some embodiments, the COX-2 inhibitor or isomer, pharmaceutically acceptable salt, ester, or prodrug thereof and the immunosuppressive agent are administered substantially simultaneously, and in alternative embodiments, they are administered sequentially.

In some embodiments, the subject has, or is at risk of developing a neoplasia disorder, for example a tumor growth, for example a malignant tumor growth or a benign tumor growth. In some embodiments, the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

In particular embodiments, the tumor or malignant growth is a colorectal cancer, for example but not limited to hereditary colorectal cancer, familial adenomautous polyposis (FAP), hereditary nonpolyposis colorectal cancer (HNPCC).

In further embodiments, the malignant growth is a viral-related cancer, for example but not limited to cervical cancer, T-cell leukemia, lymphoma, and Kaposi's sarcoma. In further embodiments, the subject has, or is at risk of developing a neoplasia disorder such as a benign tumor growth, for example a benign in a location selected from the group consisting of, but not limited to, the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis. In some embodiments, the benign tumor growth is a fibroid tumor, an endometriosis, or a cyst.

In some embodiments, subjects amenable to the methods and administration of the compositions as disclosed herein are to subjects having mutations in genes resulting in colonic and extracolonic malignancies, for example but not limited to genes are selected from the group consisting of APC gene, hMLS1, hMSH2, hMSH6. In some embodiments, a subject amenable to the methods and compositions as disclosed herein has adenomatous polyposis, colorectal polyps, sporadic adenomas, aberrant crypt foci or duodenal adenoma.

Another aspect of the present invention relates to the use of the compositions as disclosed herein for preventing and/or treating a malignancy or neoplasia disease in a subject, wherein the subject is in need of immunosuppressive therapy, for example a subject scheduled to receive or is receiving immunosuppression is a subject who is a bone marrow transplant recipient or an organ transplant recipient, or a subject having, or who is at risk of developing an auto-immune disease.

More specifically, the present invention is directed to compositions comprising NSAIDs and immunosuppressive agents for administration to subjects in need to immunosuppressive therapy, such as a subject scheduled to receive or is receiving immunosuppressive therapy. Another aspect of the present invention relates to methods to prevent and/or treat a malignancy or neoplasm subject in need of immunosuppression or a subject scheduled to receive or is receiving immununosuppression.

A further aspect of the present invention provides a composition for the treatment, prevention or inhibition of a malignancy or neoplasia disorder in a subject in need of immunosuppressive therapy, for example a subject scheduled to receive or is receiving immunosuppressive therapy, where the composition comprises a non-steroidal anti-inflammatory drug (NSAID) or a pharmaceutically acceptable salt, ester or prodrug thereof in a first amount and an immunosuppressive agent in a second amount, wherein the first amount is administered at regular or continual basis over an extended period of time and wherein the first amount together with the second amount comprises a therapeutically effective amount for immunosuppression and the treatment, prevention or inhibition of a malignancy, or neoplasia disorder in the subject.

### BRIEF DESCRIPTION OF FIGURES

FIG. 1 shows tumor volume. BALC/c mice were injected subcutaneously with MC-26 colorectal cancer cells and were either untreated (Control) or treated for 22 days with rofecoxib (R of) alone, 5-fluoroaracil+leucovorin (5-FU/LV; “standard” therapy), cyclosporine (CsA) alone or combinations of these agents.

FIG. 2 shows change (in grams) in body weight (BW) from day 0 to day 22. BALB/c mice were injected subcutaneously with MC-26 colorectal cancer cells and were either untreated (Control) or treated for 22 days with rofecoxib (R of) alone, 5-fluoroaracil+leucovorin (5-FU/LV; “standard” therapy), cyclosporine (CsA) alone or combinations of these agents.

FIG. 3 shows skin draft rate. Skin from donor B6 black mice was transplanted to recipient BALB/c mice. Animals were then randomized to receive either no treatment (control), cyclosporin (CsA) alone, rofecocib-containing chow (equivalent to a human dose of 25 mg per day), or a combination of rofecoxib (Rof) and cyclosporine.

FIG. 4 shows tumor volume. BALB/c mice were injected were injected subcutaneously with MC-26 colorectal cancer cells and were either untreated (Control) or treated for 22 days with rofecoxib (R of) alone, 5-fluoroaracil+leucovorin (5-FU/LV; “standard” therapy), cyclosporine (CsA) alone or combinations of these agents. *P<0.05, **P<0.01.

### DETAILED DESCRIPTION OF THE INVENTION

The present invention is directed to compositions comprising NSAIDs and immunosuppressive agents for administration to subjects in need to immunosuppressive therapy. Another aspect of the present invention relates to methods to prevent and/or treat a malignancy or neoplasm subject in need of immunosuppression or a subject scheduled to receive or is receiving immunosuppressive therapy.

The present disclosure provides a method for treating or preventing neoplasia disorders in a subject in need of such treatment or prevention, where the subject is in need of immunosuppression or a subject scheduled to receive or is receiving immunosuppressive therapy. The method comprises administering to a subject a therapeutically effective amount of a NSAID, for example a cyclooxygenase-2 selective inhibitor or prodrug, ester or pharmaceutically acceptable salt thereof on a regular or continued bases for an extended period of time, for example, but not limited to at least 1 month, at least 2 months, at least 3 months, at least 6 months, at least 8 months, at least the entire time the immunosuppressive agent is being administered to the subject, and all time periods in between, where the NSAID such as a cyclooxygenase-2 selective inhibitor is administered in combination with an immunosuppressant agent such as cyclosporine. In some embodiments, the NSAID is administered at regular scheduled intervals or on a continued basis for a minimum of one month and in some embodiments, the NSAID is administered to the subject at scheduled regular intervals during the same time period that the immunosuppressive agent is administered to the subject. It should be noted that the administration of the NSAID according to the methods of the present invention is different from administration of NSAIDS for other therapeutic interventions or for the treatment of acute indications, such as headache or muscle ache, when NSAIDS are typically administered for short periods of time for example for 1 or 2 days or up to a week, and administration is sporadic, in other words, administration begins on presentation of symptoms and is not a planned or scheduled administration.

Accordingly, the present inventive discovery is directed to the use of NSAIDS, for example selective inhibitors of cyclooxygenase-2 in combination with immunosuppressive agents, such as cyclosporine for the prevention or treatment of neoplasias such as cancer. More specifically, this inventive discovery relates to the use of NSAIDS such as cyclooxygenase-2 selective inhibitors or derivatives or pharmaceutically acceptable salts or prodrugs thereof in combination with immunosuppressive agents to prevent tumors.

Without wishing to be bound by theory, immunosuppressive therapy is necessary for the prevention of rejection of transplanted organs and is used in treatment of auto-immune diseases. However, one serious side effect of immunosuppression or immunosuppressive therapy is the development secondary hematological and solid malignancies. The inventors have surprisingly discovered that long-term administration of a non-steroidal anti-inflammatory drug (NSAID) in combination with an immunosuppressive agent prevents such secondary tumor growth and also prevents tissue rejection. Moreover, the long-term administration of a non-steroidal anti-inflammatory drug (NSAID) in combination with an immunosuppressive agent can also be used for the treatment of existing tumors and also prevents tissue rejection. Given the level and the number of tumors that develop as a result of immunosuppressive therapy, for example the number of solid tumors and secondary hematological tumors in subject administered an immunosuppressive agent, and the fact that NSAIDS are not typically as effective anti-cancer agents as other anti-cancer therapies such as chemotherapy or radiotherapy, it is surprising that administration of NSAIDS for example selective cyclooxygenase-2 (COX-2) inhibitors for an extended period of time can prevent tumor growth and treat existing tumors. Accordingly, the inventors have discovered a method that is a chemopreventative strategy enabling continuous administration of an immunosuppressive agent without tumor growth and/or the occurrence of hematological tumors.

As disclosed herein, using an in vivo mouse model of skin graft transplantation, the inventors have surprisingly discovered that tumor growth was prevented in immunosuppressed mice having skin graft transplantation when mice were administrated a combination of NSAIDs, such as rofecoxib and an immunosuppressive agent such as cyclosporin for extended periods of time, while maintaining immunosuppression to prevent skin graft rejection, whereas tumor growth occurred in skin graft transplantation mice administered either immunosuppression alone or NSAIDs alone.

Accordingly, the inventors have surprisingly discovered a method for the prevention and/or treatment of malignancies and neoplasms such as cancer in a subject in need of immunosuppression or subjects receiving or scheduled to receive immunosuppression, such as a transplant recipient and/or a subject with, or at risk of developing an auto-immune disease using combination therapy comprising an NSAID and an immunosuppressive agent.

The inventors' discovery that continued administration of NSAIDs such as COX-2 inhibitors for an extended period of time can prevent or inhibit tumor formation in immunosuppressed subjects is surprising given the fact that tumors typically do not respond well to the use of NSAIDs as compared with conventional anti-cancer therapies such as chemotherapeutic agents, radiotherapy or surgical resection. Further, given the amount and the number of tumors or secondary hematological tumors that develop as a side-effect of administration of immunosuppression, it is surprising that regular or continued administration of a NSAID such as a COX-2 inhibitor for an extended period of time is effective at reducing tumor formation.

### DEFINITION

As used herein, the term “neoplasia” refers to any new or abnormal growth of cells, as well as to diseases related to neoplasia.

By the term “colon” is meant that part of the large intestine that extends from 320 the cecum to the rectum. The cecum is the blind pouch in which the large intestine begins and into which the ileum opens from one side.

By the term “NSAID” is used interchangeably herein with “non-steroidal anti-inflammatory drug” and refers to any drug classified as having non-steroidal anti-inflammatory properties. NSAIDS are drugs with analgesic, antipyretic and anti-inflammatory effects, and function to reduce pain, fever and inflammation. The term “non-steroidal” is used to distinguish these drugs from steroids, which (among a broad range of other effects) have a similar eicosanoid-depressing, anti-inflammatory action. A NSAID acts by impairing prostaglandin synthesis. The term “NSAID” is intended to be interpreted broadly and is not limited in terms of chemical composition, for example the term NSAID encompasses, for example, Salicylates, Arylalkanoic acids, 2-Arylpropionic acids (profens), N-Arylanthranilic acids (fenamic acids), Pyrazolidine derivatives, Oxicams, COX-2 Inhibitors, Sulphonanilides, non-selective COX inhibitors, COX1/COX2 inhibitors and other NSAIDS.

By the term “immunosuppressive agent” it is meant any composition capable of suppressing the immune system. One example of such a composition is cyclosporine.

The term “COX-2” or “COX-2” or “cycloxygenase-2” are used interchangeably herein and refer to the gene transcript or protein encoded by the COX-2 gene. For reference purposes only, the human COX-2 gene can be identified by GenBank Sequence Identification number NM_{—}000963. COX-2 is also known by the alias of prostaglandin-endoperoxide synthase 2.

As used herein, the term “cyclosporine” includes analogs, hydrolysis products, metabolites, and precursors thereof unless the context precludes it. Cyclosporine analogs, hydrolysis products, metabolites, or precursors, and methods of synthesizing such compounds are disclosed herein under the section entitled “Immunosuppressive agents”.

By the term “treat” as in “treat a subject,” is meant to give medical aid to such subject especially, for the purposes of preventing the development of, or preventing the worsening of an undesired physiological or medical condition, or for the purposes of ameliorating such condition in such subject, either human or animal. Unless otherwise stated, the term “treat” is not limited to any particular length of time or to any particular level of dose.

Subjects in need of treatment according to the method of the invention include subjects scheduled to receive or subjects receiving immunosuppressive therapy, such as subjects that have received or are about to receive transplanted organs or subjects with a risk of developing, or having, an autoimmune disease. When a subject receives an organ transplant, the subject is typically administered immunosuppressive therapy in order to prevent rejection of the transplanted organ. A serious adverse effect of such immunosuppressive treatments is the development of secondary hematological and solid malignancies. The method of treatment as disclosed herein combines an immunosuppressive agent in conjunction with administration of a NSAID for an extended period of time, thereby enabling continued immunosuppressive therapy without increasing the risk of developing malignancies.

### NSAIDS

Non-steroidal anti-inflammatory drugs (NSAIDs) non-selectively inhibit at least one cyclooxygenase enzyme (such as COX-1 and/or COX-2 and/or COX-3 enzyme) and consequently can prevent, inhibit, or abolish the effects of prostaglandins. Increasing evidence shows that NSAIDs can inhibit the development of cancer in both experimental animals and in humans, can reduce the size of established tumors, and can increase the efficacy of cytotoxic cancer chemotherapeutic agents. However, treatment with NSAIDs are limited by toxicity to normal tissue, particularly by ulcerations and bleeding in the gastrointestinal tract, ascribed to the inhibition of Cox-1. Recently developed selective COX-2 inhibitors exert potent anti-inflammatory activity but cause fewer side effects. Compounds which selectively inhibit cyclooxygenase-2 and are useful in the methods and compositions as disclosed herein have been described in U.S. Pat. Nos. 5,380,738; 5,344,991; 5,393,790; 5,434,178; 5,474,995; 5,510,368 and WO documents WO96/06840, WO96/03388, WO96/03387, WO96/19469, WO96/25405, WO95/15316, WO94/15932, WO94/27980, WO95/00501, WO94/13635, WO94/20480, and WO94/26731 which are incorporated herein by reference in their entirety.

Additional COX-2 inhibitors have been described for the treatment of cancer (WO98/16227) and for the treatment of tumors (EP 927,555) which is incorporated herein in its entirety by reference. Celecoxib, a specific inhibitor of COX-2, exerted a potent inhibition of fibroblast growth factor-induced corneal angiogenesis in rats. (Masferrer et al., Proc. Am. Assoc. Cancer Research 1999, 40, 396), and several COX-2 inhibitors have been described for the treatment of cancer, tumors and neoplasia, for example FR 27 71 005 describes compositions containing a cyclooxygenase-2 inhibitor and N-methyl-d-asparate (NMDA) antagonist used to treat cancer and other diseases. WO 99/18960 which is incorporated herein in its entirety by reference describes a combination comprising a cyclooxygenase-2 inhibitor (iNOS) that can be used to treat colorectal and breast cancer. WO 98/41511 describes 5-(4-sulphunyl-phenyl)-pyridazinone derivatives used for treating cancer. WO 98/41516 describes (methylsulphonyl)phenyl-2-(5H)-furanone derivatives that can be used in the treatment of cancer. WO 98/16227 describes the use of cyclooxygenase-2 inhibitors in the treatment or prevention of neoplasia. WO 97/36497 describes a combination comprising a cyclooxygenase-2 inhibitor and a 5-lipoxygenase inhibitor useful in treating cancer. WO 97/11701 describes a combination comprising of a cyclooxygenase-2 inhibitor and a leukotriene B4 receptor antagonist useful in treating colorectal cancer. WO 97/29774 describes the combination of a cyclooxygenase-2 inhibitor and prostaglandin or antiulcer agent useful in treating cancer. WO 96/03385 describes 3,4-Di substituted pyrazole compounds given alone or in combination with NSAIDs, steroids, 5-LO inhibitors, LTB4 antagonists, or LTA4 hydrolase inhibitors that can be useful in the treatment of cancer. WO 98/16227 describes a method of using cyclooxygenase-2 inhibitors in the treatment and prevention of neoplasia.

Other NSAIDS useful in the methods and compositions as disclosed herein are, for example but not limited to aceclofenac, acetaminophen, alminoprofen, amfenac, aminopropylon, amixetrine, aspirin, apazone, benoxaprofen, bromfenac, bufexamac, carprofen, celecoxib, choline, salicylate, cinchophen, cinmetacin, clopriac, clometacin, diclofenac, diflunisal, etodolac, etoricoxib, flubiprofen, fenoprofen, fenamates such as mefenamic acid flurbiprofen, ibuprofen, indomethacin, indoprofen, ketoprofen, ketorolac, mazipredone, meclofenamate, nabumetone, naproxen, paracoxib, piroxicam, pirprofen, propionic acids, pyrazolones such as phenylbutazone, rofecoxib, sulindac, tolfenamate, tolmetin, lumiracoxib, etoricoxib and valdecoxib.

Other NSAIDS useful in the methods and compositions as disclosed herein are, for example but not limited to Arylalkanoic acids such as, Diclofenac, Aceclofenac, Acemetacin, Bromfenac, Etodolac, Indometacin, Nabumetone, Sulindac, Tolmetin; 2-Arylpropionic acids (profens) such as Ibuprofen, Carprofen, Fenbufen, Fenoprofen, Flurbiprofen, Ketoprofen, Ketorolac, Loxoprofen, Naproxen, Tiaprofenic acid, Suprofen; N-Arylanthranilic acids (fenamic acids) such as Mefenamic acid and Meclofenamic acid; Pyrazolidine derivatives such as for example but not limited to, Phenylbutazone, Azapropazone, Metamizole, Oxyphenbutazone, Sulfinprazone; Oxicams such as for example but not limited to, Piroxicam, Lornoxicam, Meloxicam, Tenoxicam; COX-2 Inhibitors, such as for example but not limited to, Celecoxib, Etoricoxib, Lumiracoxib, Paracoxib, Rofecoxib, Valdecoxib; Sulphonanilides such as Nimesulide, and other NSAIDS such as, Licofelone and Omega-3 Fatty Acids.

In alternative embodiments, useful in the compositions and methods as disclosed herein are non-selective COX-1/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin), COX-2 inhibitors (such as meloxicam, celecoxib, rofecoxib, valdecoxib and etoricoxib) low dose methotrexate, lefinomide, ciclesonide, hydroxychloroquine, d-penicillamine, auranofin or parenteral or oral gold.

In further embodiments, useful in the compositions and methods as disclosed herein are Suitable agents to be used in combination include standard non-steroidal anti-inflammatory agents such as analgesics and intraarticular therapies such as corticosteroids and hyaluronic acids such as hyalgan and synvisc and P2X7 receptor antagonists.

COX-2 specific inhibitors prevent angiogenesis and tumor growth in experimental animals (Rozic J G et al., 2001, Int J Cancer, 93:497-506; Liu X H et al., 2000, J. Urol, 164:820-5), but their efficacy for treatment of neoplasia and tumors as used in combination with immunosuppressive agents has not been demonstrated.

In an embodiment of the present invention, any cyclooxygenase-2 selective inhibitor or isomer, pharmaceutically acceptable salt, ester, or prodrugs thereof that meets the criteria described below can be used, along with an immunosuppressant agent, for example cyclosporine, cyclosporine analog, cyclosporine hydrolysis product, cyclosporine metabolite or cyclosporine precursor as described below, in the subject inventive method.

As used herein, the term “cyclooxygenase-2 inhibitor”, embraces compounds which selectively inhibit cyclooxygenase-2 over cyclooxygenase-1, and also includes pharmaceutically acceptable salts of those compounds.

In practice, the selectivity of a COX-2 inhibitor varies depending upon the condition under which the test is performed and on the inhibitors being tested. However, for the purposes of this specification, the selectivity of a COX-2 inhibitor can be measured as a ratio of the in vitro or in vivo IC50 value for inhibition of Cox-1, divided by the IC50 value for inhibition of COX-2 (Cox-1 IC50/COX-2 IC50). A COX-2 selective inhibitor is any inhibitor for which the ratio of Cox-1 IC50 to COX-2 IC50 is greater than 1, preferably greater than 1.5, more preferably greater than 2, even more preferably greater than 5, yet more preferably greater than 10, still more preferably greater than 50, and more preferably still greater than 100.

As used herein, the term “IC50” refers to the concentration of a compound that is required to produce 50% inhibition of cyclooxygenase activity. Preferred cyclooxygenase-2 selective inhibitors of the present invention have a cyclooxygenase-2 IC50 of less than about 5 μM, more preferred of less than about 1 μM.

In some embodiments, preferred cycloxoygenase-2 selective inhibitors have a cyclooxygenase-1 IC50 of greater than about 1 μM, and more preferably of greater than 20 μ]M. Such preferred selectivity may indicate an ability to reduce the incidence of common NSAID-induced side effects. In another embodiment, a COX-2 inhibitor or isomer, pharmaceutically acceptable salt, ester, or prodrug thereof has a selectivity ratio of COX-2 inhibition to Cox-1 inhibition of at least about 1.5, and more preferably of at least about 100.

Also included within the scope of the present invention are compounds that act as prodrugs of cyclooxygenase-2-selective inhibitors. As used herein in reference to COX-2 selective inhibitors, the term “prodrug” refers to a chemical compound that is converted into an active COX-2 selective inhibitor by metabolic processes within the body. One example of a prodrug for a COX-2 selective inhibitor is paracoxib, which is a therapeutically effective prodrug of the tricyclic cyclooxygenase-2 selective inhibitor valdecoxib, as disclosed in U.S. Pat. No. 5,932,598, which is incorporated herein in its entirety by reference. In one embodiment, a COX-2 selective inhibitor prodrug is sodium paracoxib.

In still more embodiments of the invention, a COX-2 inhibitor useful in the methods and compositions as disclosed herein can be a tricyclic cyclooxygenase-2 selective inhibitor for example, but not limited to celecoxib (B-18), valdecoxib (B-19), deracoxib (B-20), rofecoxib (B-21), etoricoxib (MK-663; B-22), JTE-522 (B-23), or an isomer, a pharmaceutically acceptable salt, ester, or prodrug thereof.

Examples of tricyclic COX-2 selective inhibitors useful in the methods and compostions as disclosed herein are disclosed in U.S. Patent Application 2003/0013739 which is incorporated herein by reference, such as B-18 Ash, celecoxib (B-19), valdecoxib (B-20), deracoxib (B-21), rofecoxib (B-22) etoricoxib (B-23), JTE-522. In some embodiments of the present invention, the COX-2 selective inhibitor, when used in combination with an immunosuppressant agent is selected from the group consisting of celecoxib, rofecoxib and etoricoxib.

In one embodiment, the cyclooxygenase-2 selective inhibitor of the present invention is, for example, the COX-2 selective inhibitor [2-(2,4-Dichloro-6-ethyl-3,5-dimethyl-phenylamino)-5-propyl-phenyl]-acetic acid, or an isomer or pharmaceutically acceptable salt, ester, or prodrug thereof. In another embodiment of the invention the cyclooxygenase-2 selective inhibitor can be the COX-2 selective inhibitor RS 57067 or 6-[5-(4-chlorobenzoyl)-1,4-dimethyl-1H-pyrrol-2-yl]methyl-3(2H)-pyridazinone (CAS registry number 179382-91-3), or an isomer, a pharmaceutically acceptable salt, or prodrug thereof. In another embodiment, the cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having a structure shown by general Formula (I) in U.S. Patent Application 2003/0013739, which is specifically incorporated herein by reference, or an isomer of pharmaceutically acceptable salt, ester of a prodrug of, or derivatives or analogues of Formula (I) as disclosed in Table 1B in U.S. Patent Application 2003/0013739, including the diastereomers, enantiomers, racemates, tautomers, salts, esters, amides and prodrugs thereof. Furthermore, benzopyran COX-2 selective inhibitors useful in the practice of the present invention are shown as Formula (I) in U.S. Patent Application 2003/0013739 and are also disclosed in U.S. Pat. Nos. 6,034,256 and 6,077,850 which are incorporated herein by reference in their entirety, or an isomer, a pharmaceutically acceptable salt, ester, or prodrug thereof.

In one embodiment, a cyclooxygenase-2 selective inhibitor useful in connection with the methods and compositions of the present invention is N-(2-cyclohexyloxynitrophenyl)-methane sulfonamide (NS-398) also known as B-26 as disclosed in U.S. Patent application 2003/0013739 which is incorporated herein by reference. Applications of this compound have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997) which are incorporated herein by reference.

Other useful cyclooxygenase-2 selective inhibitors for use in the methods and compositions as disclosed herein are cyclooxygenase-2 inhibitor derivatives and cyclooxygenase-2 inhibitor isomers, such as for example, but not limited to: 6-chloro-2-trifluoromethyl-2H-1-benzopyran-3-carboxylic acid (B-27); 6-chloro-7-methyl-2-trifluoromethyl-2H-1-benzopyran-3-carboxylic acid (B-28); 8-(1-methylethyl)-2-trifluoromethyl-2H-11 benzopyran-3-carboxylic acid (B-29); 6-chloro-8-(1-methylethyl)-2-trifluoromethyl-2H-1-benzopyran-3-carboxylic acid (B-30); 2-trifluoromethyl-3H-naphtho[2,1-b]pyran-3-carboxylic acid (B-31); 7-(1,1-dimethylethyl)-2-trifluoromethyl-2H-1-benzopyran-3-carboxylic acid (B-32); 6-bromo-2-trifluoromethyl-2H-1-benzopyran-3-carboxylic acid (B-33); 8-chloro-2-trifluoromethyl-2H-1-benzopyran-3-carboxylic acid (B-34); 6-trifluoromethoxy-2-trifluoromethyl-2H-1-benzopyran-3-carboxylic acid (B-35); 5,7-dichloro-2-trifluoromethyl-2H-1-benzopyran-3-carboxylic acid (B-36); 8-phenyl-2-trifluoromethyl-2H-1-benzopyran-3-carboxylic acid (B-37); 7,8-dimethyl-2-trifluoromethyl-2H-1-benzopyran-3-carboxylic acid (B-38); 6,8-bis(dimethylethyl)-2-trifluoromethyl-2H-1-benzopyran-3-carboxylic acid (B-39); 7-(1-methylethyl)-2-trifluoromethyl-2H-1-benzopyran-3-carboxylic acid (B-40); 7-phenyl-2-trifluoromethyl-2H-1-benzopyran-3-carboxylic acid (B-41); 6-chloro-7-ethyl-2-trifluoromethyl-2H-1-benzopyran-3-carboxylic acid (B-42); 6-chloro-8-ethyl-2-trifluoromethyl-2H-1-benzopyran-3-carboxylic acid (B-43); 6-chloro-7-phenyl-2-trifluoromethyl-2H-1-benzopyran-3-carboxylic acid (B-44); 6,7-dichloro-2-trifluoromethyl-2H-1-benzopyran-3-carboxylic acid (B-45); 6,8-dichloro-2-trifluoromethyl-2H-1-benzopyran-3-carboxylic acid (B-46); 6-chloro-8-methyl-2-trifluoromethyl-2H-1-benzopyran-3-carboxylic acid (B-47); 8-chloro-6-methyl-2-trifluoromethyl-2H-1-benzopyran-3-carboxylic acid (B-48) 8-chloro-6-methoxy-2-trifluoromethyl-2H-1-benzopyran-3-carboxylic acid (B-49); 6-bromo-8-chloro-2-trifluoromethyl-2H-1-benzopyran-3-carboxylic acid (B-50); 8-bromo-6-fluoro-2-trifluoromethyl-2H-1-benzopyran-3-carboxylic acid (B-51); 8-bromo-6-methyl-2-trifluoromethyl-2H-1-benzopyran-3-carboxylic acid (B-52); 8-bromo-5-fluoro-2-trifluoromethyl-2H-1-benzopyran-3-carboxylic acid (B-53); 6-chloro-8-fluoro-2-trifluoromethyl-2H-1-benzopyran-3-carboxylic acid (B-54); 6-bromo-8-methoxy-2-trifluoromethyl-2H-1-benzopyran-3-carboxylic acid (B-55); 6-[[(phenylmethyl)amino]sulfonyl]-2-trifluoromethyl-2H-1-benzopyran-3-carboxylic acid (B-56); 6-[(dimethylamino)sulfonyl]-2-trifluoromethyl-2H-1-benzopyran-3-carboxylic acid (B-57); 6-[(methylamino)sulfonyl]-2-trifluoromethyl-2H-1-benzopyran-3-carboxylic acid (B-58); 6-[(4-morpholino)sulfonyl]-2-trifluoromethyl-2H-1-benzopyran-3-carboxylic acid (B-59); 6-[(1,1-dimethylethyl)aminosulfonyl]-2-trifluoromethyl-2H-1-benzopyran-3-carboxylic acid (B-60); 6-[(2-methylpropyl)aminosulfonyl]-2-trifluoromethyl-2H-1-benzopyran-3-carboxylic acid (B-61); 6-methylsulfonyl-2-trifluoromethyl-2H-1-benzopyran-3-carboxylic acid (B-62); 8-chloro-6-[[(phenylmethyl)amino]sulfonyl]-2-trifluoromethyl-2H-1-benzopyran-3-carboxylic acid (B-63); 6-phenylacetyl-2-trifluoromethyl-2H-1-benzopyran-3-carboxylic acid (B-64); 6,8-dibromo-2-trifluoromethyl-2H-1-benzopyran-3-carboxylic acid (B-65) 8-chloro-5,6-dimethyl-2-trifluoromethyl-2H-1-benzopyran-3-carboxylic acid (B-66); 6,8-dichloro-(S)-2-trifluoromethyl-2H-1-benzopyran-3-carboxylic acid (B-67); 6-benzylsulfonyl-2-trifluoromethyl-2H-1-benzopyran-3-carboxylic acid (B-68); 6-[[N-(2-furylmethyl)amino]sulfonyl]-2-trifluoromethyl-2H-1-benzopyran-3-carboxylic acid (B-69); 6-[[N-(2-phenylethyl)amino]sulfonyl]-2-trifluoromethyl-2H-1-benzopyran-3-carboxylic acid (B-70); 6-iodo-2-trifluoromethyl-2H-1-benzopyran-3-carboxylic acid (B-71); 7-(1,1-dimethylethyl)-2-pentafluoroethyl-2H-1-benzopyran-3-carboxylic acid (B-72); 6-chloro-2-trifluoromethyl-2H-1-benzothiopyran-3-carboxylic acid (B-73); 3-[(3-Chloro-phenyl)-(4-methanesulfonyl-phenyl)-methylene]-dihydro-furan-2-one or BMS-347070 (B-74); 8-acetyl-3-(4-fluorophenyl)-2-(4-methylsulfonyl)phenyl-imidazo[1,2-a]pyridine (B-75); 5,5-dimethyl-4-(4-methylsulfonyl)phenyl-3-phenyl-2-(5H)-furanone (B-76); 5-(4-fluorophenyl)-1-[4-(methylsulfonyl)phenyl]-3-(trifluoromethyl)pyrazole (B-77); 4-(4-fluorophenyl)-5-[4-(methylsulfonyl)phenyl]-1-phenyl-3-(trifluoromethyl)pyrazole (B-78); 4-(5-(4-chlorophenyl)-3-(4-methoxyphenyl)-1H-pyrazol-1-yl)benzenesulfonamide (B-79); 4-(3,5-bis(4-methylphenyl)-1H-pyrazol-1-yl)benzenesulfonamide (B-80); 4-(5-(4-chlorophenyl)-3-phenyl-1H-pyrazol-1-yl)benzenesulfonamide (B-81); 4-(3,5-bis(4-methoxyphenyl)-1H-pyrazol-1-yl)benzenesulfonamide (B-82); 4-(5-(4-chlorophenyl)-3-(4-methylphenyl)-1H-pyrazol-1-yl)benzenesulfonamide (B-83); 4-(5-(4-chlorophenyl)-3-(4-nitrophenyl)-1H-pyrazol-1-yl)benzenesulfonamide (B-84); 4-(5-(4-chlorophenyl)-3-(5-chloro-2-thienyl)-1H-pyrazol-1-yl)benzenesulfonamide (B-85); 4-(4-chloro-3,5-diphenyl-1H-pyrazol-1-yl)benzenesulfonamide (B-86); 4-[5-(4-chlorophenyl)-3-(trifluoromethyl)-1H-pyrazol-1-yl]benzenesulfonamide (B-87); 4-[5-phenyl-3-(trifluoromethyl)-1H-pyrazol-1-yl]benzenesulfonamide (B-88); 4-[5-(4-fluorophenyl)-3-(trifluoromethyl)-1H-pyrazol-1-yl]benzenesulfonamide (B-89); 4-[5-(4-methoxyphenyl)-3-(trifluoromethyl)-1H-pyrazol-1-yl]benzenesulfonamide (B-90); 4-[5-(4-chlorophenyl)-3-(difluoromethyl)-1H-pyrazol-1-yl]benzenesulfonamide (B-91); 4-[5-(4-methylphenyl)-3-(trifluoromethyl)-1H-pyrazol-1-yl]benzenesulfonamide (B-92); 4-[4-chloro-5-(4-chlorophenyl)-3-(trifluoromethyl)-1H-pyrazol-1-yl]benzenesulfonamide (B-93); 4-[3-(difluoromethyl)-5-(4-methylphenyl)-1H-pyrazol-1-yl]benzenesulfonamide (B-94); 4-[3-(difluoromethyl)-5-phenyl-1H-pyrazol-1-yl]benzenesulfonamide (B-95); 4-[3-(difluoromethyl)-5-(4-methoxyphenyl)-1H-pyrazol-1-yl]benzenesulfonamide (B-96); 4-[3-cyano-5-(4-fluorophenyl)-1H-pyrazol-1-yl]benzenesulfonamide (B-97); 4-[3-(difluoromethyl)-5-(3-fluoro-4-methoxyphenyl)-1H-pyrazol-1-yl]benzenesulfonamide (B-98) 4-[5-(3-fluoro-4-methoxyphenyl)-3-(trifluoromethyl)-1H-pyrazol-1-yl]benzenesulfonamide (B-99); 4-[4-chloro-5-phenyl-1H-pyrazol-1-yl]benzenesulfonamide (B-100); 4-5-(4-chlorophenyl)-3-(hydroxymethyl)-1H-pyrazol-1-yl]benzenesulfonamide (B-101); 4-[5-(4-(N,N-dimethylamino)phenyl)-3-(trifluoromethyl)-1H-pyrazol-1-yl]benzenesulfonamide (B-102); 5-(4-fluorophenyl)-6-[4-(methylsulfonyl)phenyl]spiro[2.4]hept-5-ene (B-103); 4-[6-(4-fluorophenyl)spiro[2.4]hept-5-en-5-yl]benzenesulfonamide (B-104); 6-(4-fluorophenyl)-7-[4-(methylsulfonyl)phenyl]spiro[3.4]oct-6-ene (B-105); 5-(3-chloro-4-methoxyphenyl)-6-[4-(methylsulfonyl)phenyl]spiro[2.4]hept-5-ene (B-106); 4-[6-(3-chloro-4-methoxyphenyl)spiro[2.4]hept-5-en-5-yl]benzenesulfonamide (B-107); 5-(3,5-dichloro-4-methoxyphenyl)-6-[4-(methylsulfonyl)phenyl]spiro[2.4]hept-5-ene (B-108); 5-(3-chloro-4-fluorophenyl)-6-[4-(methylsulfonyl)phenyl]spiro[2.4]hept-5-ene (B-109); 4-[6-(3,4-dichlorophenyl)spiro[2.4]hept-5-en-5-yl]benzenesulfonamide (B-110); 2-(3-chloro-4-fluorophenyl)-4-(4-fluorophenyl)-5-(4-methylsulfonylphenyl)thiazole (B-111); 2-(2-chlorophenyl)-4-(4-fluorophenyl)-5-(4-methylsulfonylphenyl)thiazole (B-112); 5-(4-fluorophenyl)-4-(4-methylsulfonylphenyl)-2-methylthiazole (B-113); 4-(4-fluorophenyl)-5-(4-methylsulfonylphenyl)-2-trifluoromethylthiazole (B-114); 4-(4-fluorophenyl)-5-(4-methylsulfonylphenyl)-2-(2-thienyl)thiazole (B-115); 4-(4-fluorophenyl)-5-(4-methylsulfonylphenyl)-2-benzylaminothiazole (B-116); 4-(4-fluorophenyl)-5-(4-methylsulfonylphenyl)-2-(1-propylamino)thiazole (B-117); 2-[(3,5-dichlorophenoxy)methyl)-4-(4-fluorophenyl)-5-[4-(methylsulfonyl)phenyl]thiazole (B-118); 5-(4-fluorophenyl)-4-(4-methylsulfonylphenyl)-2-trifluoromethylthiazole (B-119); 1-methylsulfonyl-4-[1,1-dimethyl-4-(4-fluorophenyl)cyclopenta-2,4-dien-3-yl]benzene (B-120); 4-[4-(4-fluorophenyl)-1,1-dimethylcyclopenta-2,4-dien-3-yl]benzenesulfonamide (B-121); 5-(4-fluorophenyl)-6-[4-(methylsulfonyl)phenyl]spiro[2.4]hepta-4,6-diene (B-122); 4-[6-(4-fluorophenyl)spiro[2.4]hepta-4,6-dien-5-yl]benzenesulfonamide (B-123); 6-(4-fluorophenyl)-2-methoxy-5-[4-(methylsulfonyl)phenyl]-pyridine-3-carbonitrile (B-124); 2-bromo-6-(4-fluorophenyl)-5-[4-(methylsulfonyl)phenyl]-pyridine-3-carbonitrile (B-125); 6-(4-fluorophenyl)-5-[4-(methylsulfonyl)phenyl]-2-phenyl-pyridine-3-carbonitrile (B-126); 4-[2-(4-methylpyridin-2-yl)-4-(trifluoromethyl)-H-imidazol-1-yl]benzenesulfonamide (B-127); 4-[2-(5-methylpyridin-3-yl)-4-(trifluoromethyl)-1H-imidazol-1-yl]benzenesulfonamide (B-128); 4-[2-(2-methylpyridin-3-yl)-4-(trifluoromethyl)-1H-imidazol-1-yl]benzenesulfonamide (B-129); 3-[1-[4-(methylsulfonyl)phenyl]-4-(trifluoromethyl)-1H-imidazol-2-yl]pyridine (B-130); 2-[1-[4-(methylsulfonyl)phenyl-4-(trifluoromethyl)-1H-imidazol-2-yl]pyridine (B-131); 2-methyl-4-[1-[4-(methylsulfonyl)phenyl-4-(trifluoromethyl)-1H-imidazol-2-yl]pyridine (B-132); 2-methyl-6-[1-[4-(methylsulfonyl)phenyl-4-(trifluoromethyl)-1H-imidazol-2-yl]pyridine (B-133); 4-[2-(6-methylpyridin-3-yl)-4-(trifluoromethyl)-1H-imidazol-1-yl]benzenesulfonamide (B-134); 2-(3,4-difluorophenyl)-1-[4-(methylsulfonyl)phenyl]-4-(trifluoromethyl)-1H-imidazole (B-135); 4-[2-(4-methylphenyl)-4-(trifluoromethyl)-1H-imidazol-1-yl]benzenesulfonamide (B-136); 2-(4-chlorophenyl)-1-[4-(methylsulfonyl)phenyl]-4-methyl-1H-imidazole (B-137); 2-(4-chlorophenyl)-1-[4-(methylsulfonyl)phenyl]-4-phenyl-1H-imidazole (B-138); 2-(4-chlorophenyl)-4-(4-fluorophenyl)-1-[4-(methylsulfonyl)phenyl]-1H-imidazole (B-139); 2-(3-fluoro-4-methoxyphenyl)-1-[4-(methylsulfonyl)phenyl-4-(trifluoromethyl)-1H-imidazole (B-140); 1-[4-(methylsulfonyl)phenyl]-2-phenyl-4-trifluoromethyl-1H-imidazole (B-141); 2-(4-methylphenyl)-1-[4-(methylsulfonyl)phenyl]-4-trifluoromethyl-1H-imidazole (B-142); 4-[2-(3-chloro-4-methylphenyl)-4-(trifluoromethyl)-1H-imidazol-1-yl]benzenesulfonamide (B-143); 2-(3-fluoro-5-methylphenyl)-1-[4-(methylsulfonyl)phenyl]-4-(trifluoromethyl)-1H-imidazole (B-144); 4-[2-(3-fluoro-5-methylphenyl)-4-(trifluoromethyl)-1H-imidazol-1-yl]benzenesulfonamide (B-145); 2-(3-methylphenyl)-1-[4-(methylsulfonyl)phenyl]-4-trifluoromethyl-1H-imidazole (B-146); 4-[2-(3-methylphenyl)-4-trifluoromethyl-1H-imidazol-1-yl]benzenesulfonamide (B-147); 1-[4-(methylsulfonyl)phenyl]-2-(3-chlorophenyl)-4-trifluoromethyl-1H-imidazole (B-148); 4-[2-(3-chlorophenyl)-4-trifluoromethyl-1H-imidazol-1-yl]benzenesulfonamide (B-149); 4-[2-phenyl-4-trifluoromethyl-1H-imidazol-1-yl]benzenesulfonamide (B-150); 4-[2-(4-methoxy-3-chlorophenyl)-4-trifluoromethyl-1H-imidazol-1-yl]benzenesulfonamide (B-151); 1-allyl-4-(4-fluorophenyl)-3-[4-(methylsulfonyl)phenyl]-5-(trifluoromethyl)-1H-pyrazole (B-152); 4-[1-ethyl-4-(4-fluorophenyl)-5-(trifluoromethyl)-1H-pyrazol-3-yl]benzenesulfonamide (B-153); N-phenyl-[4-(4-fluorophenyl)-3-[4-(methylsulfonyl)phenyl]-5-(trifluoromethyl)-1H-pyrazol-1-yl]acetamide (B-154); ethyl[4-(4-fluorophenyl)-3-[4-(methylsulfonyl)phenyl]-5-(trifluoromethyl)-1H-pyrazol-1-yl]acetate (B-155); 4-(4-fluorophenyl)-3-[4-(methylsulfonyl)phenyl]-1-(2-phenylethyl)-1H-pyrazole (B-156); 4-(4-fluorophenyl)-3-[4-(methylsulfonyl)phenyl]-1-(2-phenylethyl)-5-(trifluoromethyl)pyrazole (B-157); 1-ethyl-4-(4-fluorophenyl)-3-[4-(methylsulfonyl)phenyl]-5-(trifluoromethyl)-1H-pyrazole (B-158); 5-(4-fluorophenyl)-4-(4-methylsulfonylphenyl)-2-trifluoromethyl-1H-imidazole (B-159); 4-[4-(methylsulfonyl)phenyl]-5-(2-thiophenyl)-2-(trifluoromethyl)-1H-imidazole (B-160) 5-(4-fluorophenyl)-2-methoxy-4-[4-(methylsulfonyl)phenyl]-6-(trifluoromethyl)pyridine (B-161) 2-ethoxy-5-(4-fluorophenyl)-4-[4-(methylsulfonyl)phenyl]-6-(trifluoromethyl)pyridine (B-162); 5-(4-fluorophenyl)-4-[4-(methylsulfonyl)phenyl]-2-(2-propynyloxy)-6-(trifluoromethyl)pyridine (B-163); 2-bromo-5-(4-fluorophenyl)-4-[4-(methylsulfonyl)phenyl]-6-(trifluoromethyl)pyridine (B-164); 4-[2-(3-chloro-4-methoxyphenyl)-4,5-difluorophenyl]benzenesulfonamide (B-165); 1-(4-fluorophenyl)-2-[4-(methylsulfonyl)phenyl]benzene (B-166); 5-difluoromethyl-4-(4-methylsulfonylphenyl)-3-phenylisoxazole (B-167); 4-[3-ethyl-5-phenylisoxazol-4-yl]benzenesulfonamide (B-168); 4-[5-difluoromethyl-3-phenylisoxazol-4-yl]benzenesulfonamide (B-169); 4-[5-hydroxymethyl-3-phenylisoxazol-4-yl]benzenesulfonamide (B-170); 4-[5-methyl-3-phenyl-isoxazol-4-yl]benzenesulfonamide (B-171); 1-[2-(4-fluorophenyl)cyclopenten-1-yl]-4-(methylsulfonyl)benzene (B-172); 1-[2-(4-fluoro-2-methylphenyl)cyclopenten-1-yl]-4-(methylsulfonyl)benzene (B-173); 1-[2-(4-chlorophenyl)cyclopenten-1-yl]-4-(methylsulfonyl)benzene (B-174); 1-[2-(2,4-dichlorophenyl)cyclopenten-1-yl]-4-(methylsulfonyl)benzene (B-175); 1-[2-(4-trifluoromethylphenyl)cyclopenten-1-yl]-4-(methylsulfonyl)benzene (B-176); 1-[2-(4-methylthiophenyl)cyclopenten-1-yl]-4-(methylsulfonyl)benzene (B-177); 1-[2-(4-fluorophenyl)-4,4-dimethylcyclopenten-1-yl)-4-(methylsulfonyl)benzene (B-178); 4-[2-(4-fluorophenyl)-4,4-dimethylcyclopenten-1-yl]benzenesulfonamide (B-179); 1-[2-(4-chlorophenyl)-4,4-dimethylcyclopenten-1-yl]-4-(methylsulfonyl)benzene (B-180); 4-[2-(4-chlorophenyl)-4,4-dimethylcyclopenten-1-yl]benzenesulfonamide (B-181); 4-[2-(4-fluorophenyl)cyclopenten-1-yl]benzenesulfonamide (B-182); 4-[2-(4-chlorophenyl)cyclopenten-1-yl]benzenesulfonamide (B-183); 1-[2-(4-methoxyphenyl)cyclopenten-1-yl]-4-(methylsulfonyl)benzene (B-184); 1-[2-(2,3-difluorophenyl)cyclopenten-1-yl]-4-(methylsulfonyl)benzene (B-185); 4-[2-(3-fluoro-4-methoxyphenyl)cyclopenten-1-yl]benzenesulfonamide (B-186); 1-[2-(3-chloro-4-methoxyphenyl)cyclopenten-1-yl]-4-(methylsulfonyl)benzene (B-187); 4-[2-(3-chloro-4-fluorophenyl)cyclopenten-1-yl]benzenesulfonamide (B-188); 4-[2-(2-methylpyridin-5-yl)cyclopenten-1-yl]benzenesulfonamide (B-189); ethyl 2-[4-(4-fluorophenyl)-5-[4-(methylsulfonyl)phenyl]oxazol-2-yl]-2-benzyl-acetate (B-190); 2-[4-(4-fluorophenyl)-5-[4-(methylsulfonyl)phenyl]oxazol-2-yl]acetic acid (B-191); 2-(tert-butyl)-4-(4-fluorophenyl)-5-[4-(methylsulfonyl)phenyl]oxazole (B-192); 4-(4-fluorophenyl)-5-[4-(methylsulfonyl)phenyl]-2-phenyloxazole (B-193); 4-(4-fluorophenyl)-2-methyl-5-[4-(methylsulfonyl)phenyl]oxazole (B-194); 4-[5-(3-fluoro-4-methoxyphenyl)-2-trifluoromethyl-4-oxazolyl]benzenesulfonamide (B-195); 6-chloro-7-(1,1-dimethylethyl)-2-trifluoromethyl-2H-1-benzopyran-3-carboxylic acid (B-196); 6-chloro-8-methyl-2-trifluoromethyl-2H-1-benzopyran-3-carboxylic acid (B-197); 5,5-dimethyl-3-(3-fluorophenyl)-4-methylsulfonyl-2(5H)-furanone (B-198); 6-chloro-2-trifluoromethyl-2H-1-benzothiopyran-3-carboxylic acid (B-199); 4-[5-(4-chlorophenyl)-3-(trifluoromethyl)-1H-pyrazol-1-yl]benzenesulfonamide (B-200); 4-[5-(4-methylphenyl)-3-(trifluoromethyl)-1H-pyrazol-1-yl]benzenesulfonamide (B-201); 4-[5-(3-fluoro-4-methoxyphenyl)-3-(difluoromethyl)-1H-pyrazol-1-yl]benzenesulfonamide (B-202); 3-[1-[4-(methylsulfonyl)phenyl]-4-trifluoromethyl-1H-imidazol-2-yl]pyridine (B-203); 2-methyl-5-[1-[4-(methylsulfonyl)phenyl]-4-trifluoromethyl-1H-imidazol-2-yl]pyridine (B-204); 4-[2-(5-methylpyridin-3-yl)-4-(trifluoromethyl)-1H-imidazol-1-yl]benzenesulfonamide (B-205); 4-[5-methyl-3-phenylisoxazol-4-yl]benzenesulfonamide (B-206); 4-[5-hydroxymethyl-3-phenylisoxazol-4-yl]benzenesulfonamide (B-207); [2-trifluoromethyl-5-(3,4-difluorophenyl)-4-oxazolyl]benzenesulfonamide (B-208); 4-[2-methyl-4-phenyl-5-oxazolyl]benzenesulfonamide (B-209); 4-[5-(2-fluoro-4-methoxyphenyl)-2-trifluoromethyl-4-oxazolyl]benzenesulfonamide (B-210); [2-(2-chloro-6-fluoro-phenylamino)-5-methyl-phenyl]-acetic acid or COX 189 (B-211); N-(4-Nitro-2-phenoxy-phenyl)-methanesulfonamide or nimesulide (B-212); N-[6-(2,4-difluoro-phenoxy)-1-oxo-indan-5-yl]-methanesulfonamide or flosulide (B-213); N-[6-(2,4-Difluoro-phenylsulfanyl)-1-oxo-1H-inden-5-yl]-methanesulfonamide, sodium salt or L-745337 (B-214); N-[5-(4-fluoro-phenylsulfanyl)-thiophen-2-yl]-methanesulfonamide or RWJ-63556 (B-215); 3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216); (5Z)-2-amino-5-[[3,5-bis(1,1-dimethylethyl)-4-hydroxyphenyl]methylene]-4(5H)-thiazolone or darbufelone (B-217); CS-502 (B-218); LAS-34475 (B-219); LAS-34555 (B-220); S-33516 (B-221); SD-8381 (B-222); L-783003 (B-223); N-[3-(formylamino)-4-oxo-6-phenoxy-4H-1-benzopyran-7-yl]-methanesulfonamide or T-614 (B-224); D-1367 (B-225); L-748731 (B-226); (6aR,10aR)-3-(1,1-dimethylheptyl)-6a,7,10,10a-tetrahydro-1-hydroxy-6,6-dimethyl-6H-dibenzo[b,d ]pyran-9-carboxylic acid or CT3 (B-227); CGP-28238 (B-228); 4-[[3,5-bis(1,1-dimethylethyl)-4-hydroxyphenyl]methylene]dihydro-2-methyl-2H-1,2-oxazin-3(4H)-one or BF-389 (B-229); GR-253035 (B-230); 6-dioxo-9H-purin-8-yl-cinnamic acid (B-231); or S-2474 (B-232); or an isomer, a pharmaceutically acceptable salt, ester or prodrug thereof, respectively, which are disclosed in U.S. Patent Application 2003/0013739 which is incorporated herein by reference.

In some embodiments, the NSAIDS such as COX-2 inhibitors useful in the methods and compositions as disclosed herein are chromene COX-2 inhibitors, as disclosed in U.S. Patent Application 2003/0013739 which is specifically incorporated herein by reference and includes for example but not limited to B-3 6-Nitro-2-trifluoromethyl-2H-1-benzopyran-3-carboxylic acid, B-4116-Chloro-8-methyl-2-trifluoromethyl-2H-1-benzopyran-3-carboxylic acidB-512((S)-6-Chloro-7-(1,1-dimethylethyl)-2-(trifluoromethyl-2H-1-benzopyran-3-carboxylic acidB-6132-Trifluoromethyl-2H-naphtho[2,3-b]pyran-3-carboxylic acidB-7146-Chloro-7-(4-nitrophenoxy)-2-(trifluoromethyl)-2H-1-benzopyran-3-carboxylic acidB-815((S)-6,8-Dichloro-2-(trifluoromethyl)-2H-1-benzopyran-3-carboxylic acidB-9166-Chloro-2-(trifluoromethyl)-4-phenyl-2H-1-benzopyran-3-carboxylic acidB-10176-(4-Hydroxybenzoyl)-2-(trifluoromethyl)-2H-1-benzopyran-3-carboxylic acidB-11182-(Trifluoromethyl)-6-[(trifluoromethyl)thio]-2H-1-benzothiopyran-3-carboxylic acidB-12196,8-Dichloro-2-trifluoromethyl-2H-1-benzothiopyran-3-carboxylic acidB-13206-(1,1-Dimethylethyl)-2-(trifluoromethyl)-2H-1-benzothiopyran-3-carboxylic acidB-14216,7-Difluoro-1,2-dihydro-2-(trifluoromethyl)-3-quinolinecarboxylic acidB-15226-Chloro-1,2-dihydro-1-methyl-2-(trifluoromethyl)-3-quinolinecarboxylic acidB-16236-Chloro-2-(trifluoromethyl)-1,2-dihydro[1,8]naphthyridine-3-carboxylic acidB-1724((S)-6-Chloro-1,2-dihydro-2-(trifluoromethyl)-3-quinolinecarboxylic acid.

### Immunosuppressive Agents

As used herein, the term “immunosuppressive agents” includes analogs, hydrolysis products, metabolites, and precursors of an immunosuppressive agent unless the context precludes it. In some embodiments, immunosuppressive agents useful in the compositions and methods as disclosed herein can be selected from one of the following compounds: mycophenolic acid, cyclosporin, azathioprine, tacrolimus, cyclosporin A, FK506, rapamycin, leflunomide, deoxyspergualin, prednisone, azathioprine, mycophenolate mofetil, OKT3, ATAG or mizoribine.

Cyclosporins comprise a class of structurally distinct, cyclic, poly-N-methylated undecapeptides, generally possessing immunosuppressive, anti-inflammatory, anti-viral and/or anti-parasitic activity, each to a greater or lesser degree. The first of the cyclosporins to be identified was the fungal metabolite Cyclosporin A, or Ciclosporin, and its structure is given in The Merck Index, 11th Edition; Merck & Co., Inc.; Rahway, N.J., USA (1989) under listing 2759. Later cyclosporins to be identified are cyclosporins B, C, D and G which are also listed in the Merck Index under listing 2759. A large number of synthetic analogues are also known and representative examples are disclosed in EP 296 123, EP 484 281, and GB 2222770 which are incorporated herein in their entirety by reference. Cyclosporin A and its structurally similar analogues and derivatives are generally referred to as “cyclosporins” for the purposes of this specification.

The cyclosporins are a family of, neutral, hydrophobic cyclic undecapeptides, containing a novel nine-carbon amino acid (MeBmt) at position 1 of the ring that exhibit potent immunosuppressive, antiparasitic, fungicidal, and chronic anti-inflammatory properties. The naturally occurring members of this family of structurally related compounds are produced by various fungi imperfecti. Cyclosporines A and C, are the major components. Cyclosporine A, which is discussed further below, is a particularly important member of the cyclosporin family of compounds. Twenty four minor metabolites, also oligopeptides, have been identified: Lawen et al, J. Antibiotics 42, 1283 (1989); Traber et al, Helv. Chim. Acta 70, 13 (1987); Von Wartburg and Traber Prog. Med. Chem., 25, 1 (1988).

Cyclosporin was discovered to be immunosuppressive when it was observed to suppress antibody production in mice during the screening of fungal extracts. Specifically, its suppressive effects appear to be related to the inhibition of T-cell receptor-mediated activation events. It accomplishes this by interrupting calcium dependent signal transduction during T-cell activation by inactivating calmodulin and cyclophilin, a peptidly propyl isomerase. It also inhibits lymphokine production by T-helper cells in vitro and arrests the development of mature CD8 and CD4 cells in the thymus. Other in vitro properties include inhibition of IL-2 producing T-lymphocytes and cytotoxic T-lymphocytes, inhibition of IL-2 released by activated T-cells, inhibition of resting T-lymphocytes in response to alloantigen and exogenous lymphokine, inhibition of IL-1 production, and inhibition of mitogen activation of IL-2 producing T-lymphocytes, Further evidence indicates that the above effects involve the T-lymphocytes at the activation and maturation stages.

A number of cyclosporines and analogs have been described in the patent literature and are encompassed for use in the methods and composition as disclosed herein, for example, the following cyclosporine analogues and derivatives are encompassed for use in accordance with the present invention; U.S. Pat. No. 4,108,985 which is incorporated herein by reference, issued to Ruegger, et al. on Aug. 22, 1978 entitled, “Dihydrocyclosporin C”, discloses dihydrocyclosporin C, which can be produced by hydrogenation of cyclosporin C. U.S. Pat. No. 4,117,118 which is incorporated herein by reference, issued to Harri, et al. on Sep. 26, 1978 entitled, “Organic Compounds”, discloses cyclosporins A and B, and the production thereof by fermentation. U.S. Pat. No. 4,210,581 which is incorporated herein by reference, issued to Ruegger, et al. on Jul. 1, 1980 entitled, “Organic Compounds”, discloses cyclosporin C and dihydrocyclosporin C which can be produced by hydrogenation of cyclosporin C. U.S. Pat. No. 4,220,641, which is incorporated herein by reference, issued to Traber, et al. on Sep. 2, 1980 entitled, “Organic Compounds”, discloses cyclosporin D, dihydrocyclosporin D, and isocyclosporin D. U.S. Pat. No. 4,288,431 which is incorporated herein by reference, issued to Traber, et al. on Sep. 8, 1981 entitled, “Cyclosporin Derivatives, Their Production and Pharmaceutical Compositions Containing Them”, discloses cyclosporin G, dihydrocylosporin G, and isocyclosporin G. U.S. Pat. No. 4,289,851, which is incorporated herein by reference, issued to Traber, et al. on Sep. 15, 1981 entitled, “Process for Producing Cyclosporin Derivatives”, discloses cyclosporin D, dihydrocyclosporin D, and isocyclosporin D, and a process for producing same. U.S. Pat. No. 4,384,996, which is incorporated herein by reference, issued to Bollinger, et al. on May 24, 1983 entitled “Novel Cyclosporins”, discloses cyclosporins having a β-vinylene-α-amino acid residue at the 2-position and/or a β-hydroxy-α-amino acid residue at the 8-position. The cyclosporins disclosed included either MeBmt or dihydro-MeBmt at the 1-position. U.S. Pat. No. 4,396,542, which is incorporated herein by reference, issued to Wenger on Aug. 2, 1983 entitled, “Method for the Total Synthesis of Cyclosporins, Novel Cyclosporins and Novel Intermediates and Methods for their Production”, discloses the synthesis of cyclosporins, wherein the residue at the 1-position is either MeBmt, dihydro-MeBmt, and protected intermediates. U.S. Pat. No. 4,639,434, which is incorporated herein by reference, issued to Wenger, et al on Jan. 27, 1987, entitled “Novel Cyclosporins”, discloses cyclosporins with substituted residues at positions 1, 2, 5 and 8. U.S. Pat. No. 4,681,754, which is incorporated herein by reference, issued to Siegel on Jul. 21, 1987 entitled, “Counteracting Cyclosporin Organ Toxicity”, discloses methods of use of cyclosporin comprising co-dergocrine. U.S. Pat. No. 4,703,033 which is incorporated herein by reference, issued to Seebach on Oct. 27, 1987 entitled, “Novel Cyclosporins”, discloses cyclosporins with substituted residues at positions 1, 2 and 3. The substitutions at position-3 include halogen. H. Kobel and R. Traber, Directed Biosynthesis of Cyclosporins, European J. Appln. Microbiol. Biotechnol., 14, 237B240 (1982), discloses the biosynthesis of cyclosporins A, B, C, D & G by fermentation. Additional cyclosporin analogs are disclosed in U.S. Pat. No. 4,798,823, which is incorporated herein by reference, issued to Witzel, entitled, New Cyclosporin Analogs with Modified “C-9 amino acids”, which discloses cyclosporin analogs with sulfur-containing amino acids at position-1. Furthermore, European Patent Application EP1645565 and U.S. Provisional Application 60/061,360 filed Oct. 8, 1997, which is incorporated herein in its entirety discloses cyclosporine derivatives which possess enhanced efficacy and reduced toxicity over naturally occurring and other presently known cyclosporins and cyclosporine derivatives chemical and isotopic substitution of the cyclosporine A (CsA) molecule by Chemical substitution and optionally deuterium substitution of amino acid 1; and deuterium substitution at key sites of metabolism of the cyclosporine A molecule such as amino acids 1, 4, 9.

Accordingly, cyclosporine analogues and derivatives encompassed for use in the methods and compositions as disclosed herein include, but are not limited to; dihydrocyclosporin C, cyclosporins A and B, cyclosporin C and dihydrocyclosporin C; cyclosporin D, dihydrocyclosporin D, and isocyclosporin D; cyclosporin G, dihydrocylosporin G, and isocyclosporin G; cyclosporin D, dihydrocyclosporin D, and isocyclosporin D; cyclosporins having a β-vinylene-α-amino acid residue at the 2-position and/or a β-hydroxy-α-amino acid residue at the 8-position. The cyclosporins disclosed included either MeBmt or dihydro-MeBmt at the 1-position; cyclosporins, wherein the residue at the 1-position is either MeBmt, dihydro-MeBmt; cyclosporins with substituted residues at positions 1, 2, 5 and 8; cyclosporin comprising co-dergocrine; cyclosporins with substituted residues at positions 1, 2 and 3 and cyclosporin analogs with sulfur-containing amino acids at position-1.

In further embodiments, the immunosuppressive agents useful in the compositions and methods as disclosed herein are, for example calcineurin inhibitors (e.g. tacrolimus and cyclosporin A); steroids; agents which interfere with cytokine production or signalling, such as Janus Kinase (JAK) inhibitors (e.g. JAK-3 inhibitors, including 3-[(3R,4R)-4-methyl-3-[methyl-(7H-pyrrolo[2,3-d]pyrimidin-4-yl)-amino)-piperidin-1-yl]-3-oxo-propionitrile) and pharmaceutically acceptable salts, solvates or derivatives thereof; cytokine antibodies (e.g. antibodies that inhibit the interleukin-2 (IL-2) receptor, including basiliximab and daclizumab); and agents which interfere with cell activation or cell cycling, such as rapamycin.

In some embodiments, the immunosuppressant agent useful in the compositions and methods as disclosed herein is rapamycin. Rapamycin is a macrolide immunosuppressant that is produced by Streptomyces hygroscopicus and which has been found to be pharmaceutically useful in a variety of applications, particularly as an immunosuppressant for use in the treatment and prevention of organ transplant rejection and autoimmune diseases. The structure of rapamycin is given in Kesseler, H., et al.; 1993; Helv. Chim. Acta; 76: 117. Large numbers of derivatives of rapamycin have been synthesized, including for example 40-O-alkylated derivatives such as 40-O-(2-hydroxy)ethyl-rapamycin (WO 94/09010, which is incorporated herein in its entirety by reference), certain acyl and aminoacyl-rapamycins (e.g., U.S. Pat. No. 4,316,885, U.S. Pat. No. 4,650,803, and U.S. Pat. No. 5,151,413 which are incorporated herein in their entirety by reference), 27-desmethyl-rapamycin (WO 92/14737 which is incorporated herein in its entirety by reference), 26-dihydro-rapamycin (U.S. Pat. No. 5,138,051 which is incorporated herein in its entirety by reference), certain pyrazole derivatives (U.S. Pat. No. 5,164,399 which is incorporated herein in its entirety by reference), certain alkoxyester derivatives (U.S. Pat. No. 5,233,036 which is incorporated herein in its entirety by reference), and numerous others. Rapamycin and its structurally similar analogues and derivatives are termed collectively as “rapamycins” in this specification.

In some embodiments, the immunosuppressant agent useful in the compositions and methods as disclosed herein is ascomycin. Ascomycins, of which FK-506 is the best known, are another class of generally immunosuppressive macrolides. FK506 is a macrolide immunosuppressant that is produced by Streptomyces tsukubaensis No 9993. The structure of FK506 is given in the appendix to the Merck Index, as item A5. A large number of related compounds which retain the basic structure and immunological properties of FK506 are also known. These compounds are described in various publications, for example EP 184162, EP 315973, EP 323042, EP 423714, EP 427680, EP 465-426, EP 474126, WO 91/13889, WO 91/19495, EP 484936, EP 532088, EP 532089, WO 93/5059 and the like, which are incorporated herein in their entirety by reference. Ascomycin, FK-506 and their structurally similar analogues and derivatives are termed collectively “ascomycins” in this specification.

One can determine if an agent is an immunosuppressive agent using assay methods for monitoring the concentration in bodily fluids of immunosuppressant affecting gene expression, as disclosed in EP patent 750681 which is incorporated herein by reference, which describes methods for ex vivo monitoring of immunosuppressant agents e.g., cyclosporins, such as cyclosporin A or cyclosporin G; ascomycins, such as FK-506; and rapamycins, e.g., rapamycin; using a reporter gene assay, e.g., an IL-2 reporter gene assay for immunosuppressive cyclosporins and ascomycins, or a c-jun reporter gene assay for immunosuppressive rapamycins.

Furthermore, one can use methods as disclosed in WO 93/25712, which describes a method for identifying compounds capable of inducing immunosuppression by inhibiting the CD28 signal transduction pathway, or the methods disclosed in EP 519'336 which describes a chimeric gene comprising a promoter-regulator (e.g. IL-2) and a gene coding for an indicator protein (e.g. diphtheria toxin A). Williams et al. (1989) Analytical Biochemistry 176: 28-32 describes some advantages of firefly Luciferase as a reporter gene. WO 93/04203 describes a method of screening for a candidate immunosuppressant agent upon the ability of NF-AT to activate transcription.

One can further assess if a compound has immunosuppressive biological activity using an assay to measure inhibition of T-cell proliferation, such as the assay described in detail in Dumont, F. J. et al, J. Immunol. (1990) 144:251 which is incorporated by reference herein. Briefly, one can use an assay that measures T-cell proliferation in mouse T-cell cultures stimulated with ionomycin plus phorbol myristate acetate (PMA). Spleen cell suspensions from C57B1/6 mice are prepared and separated on nylon wool columns. The recovered T-cells can be suspended at 10⁶ cells/ml in complete culture medium with addition of ionomycin (250 ng/ml) and PMA (10 ng/ml), distributed in 96 well-flat bottom microculture plates at 200 μl/well. Control medium or various concentrations of test compound are added in triplicate wells at 20 μl/well, and parallel cultures were set up with exogenous IL-2 (50 units/ml). The plates are incubated as 37° C. in a humidified atmosphere of 5% CO₂-95% air for 44 hours. The cultures are then pulsed with tritiated-thymidine (2 uCi/well) for an additional 4 hour period and cells collected on fiber glass filters using a multisample harvester. Incorporated radioactivity can be measured using any method known by person of ordinary skill in the art, such as a BETAPLATE COUNTER (PHARMACIA/LKB, Piscataway, N.J.) and the mean count per minute (cpm) values of triplicate samples calculated. The percent inhibition of proliferation was calculated according to the formula: % Inhibition.=100−mean cpm experimental/mean cpm control medium/X 100. This assay is described in detail in Dumont, F. J. et al, J. Immunol. (1990) 144:251 which is incorporated herein by reference.

### Methods of Treatment

In some embodiments, the methods and compositions as disclosed herein comprising NSAIDs such as selective COX-2 inhibitors and immunosuppressive agents are administered to subjects in need of treatment or scheduled to receive or are receiving immunosuppressive therapy. In some embodiments, the subjects are in need of treatment are in need of immune suppression, for example subjects who are about to have, are having, or have received transplanted organs or bone marrow transplant or subjects who would normally be administered immunosuppression, such as organ transplant recipient in order to prevent rejection of the transplanted organ. In alternative embodiments, a subject in need of treatment is a subject in need of immunosuppression, for example a subject with an auto-immune disease, such as rheumatoid arthritis or multiple sclerosis.

As used herein, “autoimmune disease” or “auto-immune disease” are used interchangeably, or autoimmune-related disease refers to an illness that occurs when the body tissues are attacked by its own immune system. The immune system is a complex organization within the body that is designed normally to “seek and destroy” invaders of the body, including infectious agents. Subjects with autoimmune diseases frequently have unusual antibodies circulating in their blood that target their own body tissues.

Examples of autoimmune diseases include, for example but are not limited to systemic lupus erythematosus, Sjogren syndrome, Hashimoto thyroiditis, rheumatoid arthritis, juvenile (type 1) diabetes, polymyositis, scleroderma, Addison disease, vitiligo, pernicious anemia, glomerulonephritis, multiple sclerosis, and pulmonary fibrosis. Autoimmune diseases also include diseases with immunoregulatory abnormalities, such as for example a wide variety of autoimmune and chronic inflammatory diseases, including systemic lupus erythematosis, chronic rheumatoid arthritis, type I diabetes mellitus, inflammatory bowel disease, biliary cirrhosis, uveitis, multiple sclerosis, graft versus host (GVH) disease and other disorders such as Crohn's disease, ulcerative colitis, bullous pemphigoid, sarcoidosis, psoriasis, ichthyosis, and Graves opthalmopathy. Although the underlying pathogenesis of each of these conditions can be quite different, they have in common the appearance of a variety of autoantibodies and self-reactive lymphocytes, and are therefore auto-immune disease which can be treated with the compositions and methods as disclosed herein.

Autoimmune diseases are also referred to in the art as intractable diseases and allergic diseases. As used herein, “allergic disease” refers to a disease associated with allergic reaction. Specific examples include chronic bronchitis, atopic dermatitis, pollinosis (allergic rhinitis), allergic angiitis, allergic conjunctivitis, allergic gastroentelitis, allergic hepatopathy, allergic cystitis, and allergic purpura.

Without being bound by theory, as used herein the term “immune disease” refers to a disease resulting from dysfunction of the immune system, one of defense mechanisms in the body, including diseases caused by both abnormal humoral and cellular immunity. This term also includes autoimmune diseases caused by autoantibody, autosensitized lymphocyte or immune complex, as well as graft versus host disease caused by graft versus host reaction (GVH reaction) in which graft rejection occurs, as well as allergic diseases. As used herein, auto-immune disease also include “Th1-dominant autoimmune diseases” which are autoimmune disease showing increased cytokine production from Th1 cells, including IFN-y, IL-2, GM-CSF, TNF-α, and IL-3. Specific examples of such Th1-dominant autoimmune diseases include, for example but are not limited to, multiple sclerosis, insulin-dependent diabetes mellitus, Crohn's disease, uveitis, chronic rheumatism, and systemic lupus erythematosus. As used herein, the term autoimmune diseases also encompass “autoimmune diseases not known to be Th 1-dominant” which are autoimmune disease that is not known to show increased cytokine production from Th1 cells. Specific examples include scleroderma, multiple myositis, vasculitis syndrome, mixed connective tissue disease, Sjogren's syndrome, hyperthyroidism, Hashimoto's disease, myasthenia gravis, Guillain-Barre syndrome, autoimmune hepatopathy, ulcerative colitis, autoimmune nephropathy, autoimmune hematopathy, idiopathic interstitial pneumonia, hypersensitivity pneumonitis, autoimmune dermatosis, autoimmune cardiopathy, autoimmune infertility, and Behcet's disease.

One commonly known auto-immune disease is graft versus host disease caused by graft versus host reaction (GVH reaction) in which graft rejection occurs. In subjects that are recipients of organ transplantation, such as bone marrow transplantation, the host lymphocytes recognize the foreign tissue antigens and begin to produce antibodies which lead to graft rejection. Accordingly, subjects that are recipients of bone marrow transplants or organ transplants, either before transplantation, at the time of transplantation or post transplantation can be treated with the compositions and methods as disclosed herein.

Accordingly, the methods and compositions as disclosed herein comprising NSAIDs, such as COX-2 inhibitors, and immunosuppressive agents are administered to subjects in need of, or scheduled to receive immune suppression (i.e. in need of immunosuppressive therapy), for example subjects who are organ or bone marrow transplant recipients or subjects having, or at risk of developing an auto-immune disease.

In some embodiments, the methods and compositions as disclosed herein comprising NSAIDs, such as selective COX-2 inhibitors, and immunosuppressive agents are administered to a subject for the treatment and/or prevention of malignancies in a subject concurrently undergoing immunosuppression therapy. Malignancies that can be treated or prevented according to the methods and compositions as disclosed herein include, for example, but are not limited to tumor growth or tumor cell growth, including benign tumor growth and malignant tumor growth, metastasis, hematological tumors, acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, blastoma, breast cancer including benign tumor growth in the breast, bronchial gland carcinomas, capillary, carcinoids, carcinoma, carcinosarcoma, cavernous, cholangiocarcinoma, chondrosarcoma, choroid plexus papilloma/carcinoma, clear cell carcinoma, cystadenoma, cyst, ovarian cyst, endodermal sinus tumor, endometrial hyperplasia, endometrial stromal sarcoma, endometrioid adenocarcinoma, ependymal, epitheloid, Ewing's sarcoma, fibrolamellar, fibroma, fibroid tumor, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intraepithelial neoplasia, intraepithelial squamous cell neoplasia, invasive squamous cell carcinoma, large cell carcinoma, leiomyosarcoma, lentigo maligna melanomas, lipoma, malignant melanoma, malignant mesothelial tumors, medulloblastoma, medulloepithelioma, melanoma, meningeal, mesothelial, metastatic carcinoma, mucoepidermoid carcinoma, myoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, polyp, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

In some embodiments, the malignancies that can be treated or prevented using the methods and compositions as disclosed herein include, for example, malignancies in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis. Malignant growth can also include viral-related cancers, including but not restricted to cervical cancer, T-cell leukemia, lymphoma, and Kaposi's sarcoma.

In another embodiment, the malignancies are benign tumor growth in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis. In another embodiment, the benign tumor growth is a fibroid tumor, an endometriosis, or a cyst.

Additionally, the methods and treatments as disclosed herein are useful to treat subjects who are at risk of developing polyps and/or colon cancer, or subjects whom have, or been identified to be at risk of developing polyps and/or colon cancer, such as subjects with mutations in genes APC, hMLS1, hMLS2, hMSH6 as disclosed herein. In particular, subjects who have, or are at risk of developing polyp conditions or syndromes including, for example, adenomatous colonic polyps (especially of the large bowel, the precursor lesions for the vast majority of colorectal cancers), common sporadic polyps, familial adenomatous polyposis (FAP), polyposis syndromes, Gardner's Syndrome with colon polyposis, and colorectal carcinoma would benefit by treatment according to methods and compositions of the present invention. The difference between common sporadic polyps and polyposis syndromes is dramatic. Common sporadic polyp cases are characterized by relatively few polyps, each of which can usually be removed leaving the colon intact. By contrast, polyposis syndrome cases can be characterized by many (hundreds or more) of polyps literally covering the colon in some cases, making safe removal of the polyps impossible short of surgical removal of the colon.

Chemotherapeutic agents or modalities to treat refractory colorectal cancer, while associated with significant toxicity, have provided only minimal benefit in improving survival. The present invention combines the administration of an immunosuppressive agent, such as cyclosporine with the continued administration an NSAID for an extended period of time to provide a method for treating or preventing colorectal cancer. One benefit of the methods and compositions of the present invention is improved efficacy in the treatment of colorectal cancer and potentially other solid tumors without significant toxicity and the maintenance of the subject's well-being, such as continued weight gain.

Dosages can be determined by those skilled in the art. In particular, dosages can be determined by a clinician where both the NSAID such as COX-2 inhibitor and immunosuppressant such as cyclosporine, are biologically active at the same time. Stated in another way, the amount of a NSAID, such as a COX-2 inhibitor is a therapeutically effective amount admininsered for an extended period of time, for example at least 1 month, at least 2 months, at least 3 months etc. to prevent or treat a malignancy is administered with a therapeutically effective amount of an immunosuppressant such as cyclosporine to treat and/or prevent tissue rejection and/or an auto-immune disease. Further, it is noted that the ordinary skilled clinician or treating physician will know how and when to interrupt, adjust or terminate therapy in consideration of a subjects response.

### Pharmaceutical Compositions

The NSAIDS, for example cyclooxygenase-2 selective inhibitors as described herein can be referred to herein collectively as “COX-2 selective inhibitors”, “Cox-2 selective inhibitors”, or “cyclooxygenase-2 selective inhibitors” or “COX-2 inhibitors”, “Cox-2 inhibitors”, or “cyclooxygenase-2 inhibitors”.

Cyclooxygenase-2 selective inhibitors as well as immunosuppressive agents such as cyclosporine useful in the methods and compositions as disclosed herein can be supplied by any source as long as the combination of drugs is pharmaceutically acceptable. Cyclooxygenase-2 selective inhibitors and immunosuppressive agents such as cyclosporine can be isolated and purified from natural sources or can be synthesized. The combination of a cyclooxygenase-2 selective inhibitor(s) and immunosuppressive agents such as cyclosporine should be of a quality and purity that is conventional in the trade for use in pharmaceutical products.

In the methods and compositions as disclosed herein, a subject in need of immunosuppression or in need immunosuppressive therapy or a subject scheduled to receive or is receiving immunosuppressive therapy, for example a subject who is a transplant recipient or a subject with an auto-immune disease, is treated on a regular or continued bases for an extended period of time with an amount of a NSAID, such as a COX-2 selective inhibitor, and an amount of immunosuppressive agents such as cyclosporine, where the amount of the COX-2 selective inhibitor together with the amount of immunosuppressive agents such as cyclosporine is sufficient to constitute a therapeutically effective amount for treating an auto-immune disease or an organ transplant recipient, such as a subject with graft versus host disease, as well as a sufficient amount to constitute a therapeutically effective amount to prevent or treat a malignancy.

As used herein, an “effective amount” or “therapeutically effective amount” means the dose or effective amount to be administered to a subject and the frequency of administration to a subject which is sufficient to obtain a therapeutic effect as readily determined by one of ordinary skill in the art, by the use of known techniques and by observing results obtained under analogous circumstances. The dose or effective amount to be administered to a subject and the frequency of administration to a subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of a subject to be treated, and other relevant circumstances.

The phrase “therapeutically effective” indicates the capability of a combination of agents to prevent, or reduce the severity of, the disorder or its undesirable symptoms, while avoiding adverse side effects typically associated with alternative therapies. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The amounts of a NSAID, such as a COX-2 selective inhibitor, and immunosuppressive agents such as cyclosporine that are used in the methods and compositions as disclosed herein can be amounts that, together, are sufficient to constitute an effective amount for immunosuppressive treatment or therapy as well as for treatment, prevention or inhibition of a malignancy or tumor growth. In some embodiments, the amount of COX-2 selective inhibitor that is used in the compositions and methods as disclosed herein ranges from about 0.001 to about 100 milligrams per day per kilogram of body weight of a subject (mg/day/kg), and in some embodiments from about 0.05 to about 50 mg/day/kg, even more preferably from about 1 to about 20 mg/day/kg.

In some embodiments, when the COX-2 selective inhibitor comprises rofecoxib, the amount used can be within a range of from about 0.15 to about 1.0 mg/day/kg, and even more preferably from about 0.18 to about 0.4 mg/day/kg. In some embodiments, when the COX-2 selective inhibitor comprises etoricoxib, an amount used can be within a range of from about 0.5 to about 5 mg/day/kg, and even more preferably from about 0.8 to about 4 mg/day/kg. In some embodiments, when the COX-2 selective inhibitor comprises celecoxib, the amount used can be within a range of from about 1 to about 20 mg/day/kg, even more preferably from about 1.4 to about 8.6 mg/day-kg, and yet more preferably from about 2 to about 3 mg/day-kg. In some embodiments, when the COX-2 selective inhibitor comprises valdecoxib, the amount used can be within a range of from about 0.1 to about 5 mg/day-kg, and even more preferably from about 0.8 to about 4 mg/day-kg. In some embodiments, when the COX-2 selective inhibitor comprises paracoxib, the amount used can be within a range of from about 0.1 to about 5 mg/day/kg, and even more preferably from about 1 to about 3 mg/day/kg.

In terms of daily dosages, the COX-2 selective inhibitor such as the amount used can be from about 10 to about 75 mg/day, more preferably from about 12.5 to about 50 mg/day. In some embodiments, a COX-2 selective inhibitor such as etoricoxib, the amount used can be from about 50 to about 100 mg/day, more preferably from about 60 to about 90 mg/day. In some embodiments, a COX-2 selective inhibitor such as celecoxib, the amount used can be from about 100 to about 1000 mg/day, more preferably from about 200 to about 800 mg/day. In some embodiments, when the COX-2 selective inhibitor comprises valdecoxib, the amount used can be from about 5 to about 100 mg/day, more preferably from about 10 to about 60 mg/day. In some embodiments, when the COX-2 selective inhibitor comprises paracoxib, the amount used can be within a range of from about 10 to about 100 mg/day, more preferably from about 20 to about 80 mg/day.

The NSAID for example COX-2 inhibitor is administered to a subject at a frequency and dose sufficient for the NSAID, such as COX-2 inhibitor to be biologically active over an extended period of time, for example but not limited to at least one month, at least 2 months or at least 3 months etc. Preferably, the NSAID such as COX-2 inhibitor is biologically active of the entire period of time the immunosuppressant agent is administered to the subject. Accordingly, in some embodiments, the NSAID for example COX-2 inhibitor can be administered on a regular scheduled administration or it can be administered continually for example by a pump or catheter.

Administration of NSAIDS, for example administration of a COX-2 inhibitor according to the methods of the present invention is different from administration of NSAIDS, such as short term administration of NSAIDS for other therapeutic interventions or for the treatment of acute indications, such as headache or muscle ache, when NSAIDS are typically administered for short periods of time, for example for 1 or 2 days or up to a week, and administration is sporadic, in other words, administration begins on presentation of symptoms and is not a planned or scheduled administration.

The frequency of dose of the NSAID, for example COX-2 inhibitor will depend upon the half-life of NSAID, for example COX-2 inhibitor or an analog, hydrolysis product, metabolite, or precursor thereof. If the NSAID, for example COX-2 inhibitor or analog, hydrolysis product, metabolite, or precursor thereof has a short half-life (e.g. from about 2 to 10 hours) it can be necessary to give one or more doses per day. Alternatively, if the NSAID for example COX-2 inhibitor or analog, hydrolysis product, metabolite, or precursor thereof has a long half-life (e.g. from about 2 to about 15 days) it may only be necessary to give a dosage once per day, per week, or even once every 1 or 2 months. A preferred dosage rate is to administer the dosage amounts described above to a subject once per day. It will be apparent to those skilled in the art that it is possible, and perhaps desirable, to combine various times and methods of administration in the practice of the present methods.

Importantly, the NSAID for example COX-2 inhibitor is administered to the subject on a regular schedule and for an extended period of time, for example for at least one month, or at least 2 months or at least 3 months, for at least 6 months, for at least 8 months, for at least the time of administration of the immunosuppressant agent etc. In some embodiments, the amount of immunosuppressive agents such as cyclosporine that is used in combination with a COX-2 selective inhibitor for a single dosage of treatment is within range of from about 0.05 to about 2500 milligrams per kilogram of body weight per day (mg/kg/day), preferably of from about 100 to about 500 milligrams per kilogram of body weight per day (mg/kg/day), and more preferably from about 200 to about 400 milligrams per kilogram of body weight per day (mg/kg/day), and in some embodiments, from about 1 to about 50 milligrams per kilogram of body weight per day (mg/kg/day). In some embodiments, a COX-2 selective inhibitor for use in the methods and compositions as disclosed herein can exceed 2500 milligrams per kilogram of body weight per day (mg/kg/day) up to, or above the maximum tolerated dose (MTD) of the COX-2 selective inhibitor in humans. A MTD as used herein is operationally defined in toxicology as the highest daily dose of an agent that does not cause overt toxicity in humans. MTD is typically assessed by administering the agent in a ninety-day study in laboratory mice or rats, and is a dose used for longer-term safety assessment in the same species, usually lasting two years or a lifetime.

The frequency of dose will depend upon the half-life of immunosuppressive agents such as cyclosporine or an analog, hydrolysis product, metabolite, or precursor thereof. If the immunosuppressive agents such as cyclosporine or analog, hydrolysis product, metabolite, or precursor thereof has a short half-life (e.g. from about 2 to 10 hours) it can be necessary to give one or more doses per day. Alternatively, it the immunosuppressive agents such as cyclosporine or analog, hydrolysis product, metabolite, or precursor thereof has a long half-life (e.g. from about 2 to about 15 days) it may only be necessary to give a dosage once per day, per week, or even once every 1 or 2 months. A preferred dosage rate is to administer the dosage amounts described above to a subject once per day. It will be apparent to those skilled in the art that it is possible, and perhaps desirable, to combine various times and methods of administration in the practice of the present methods.

In one embodiment, the compositions as disclosed herein can be administered initially by intravenous injection to bring blood levels to a suitable level. An oral dosage form then maintains the subject's composition levels. Additionally, other forms of administration, dependent upon the subject's condition and as indicated above, can be used. The quantity to be administered will vary for a subject being treated and will be dependent on the NSAID being delivered. In some embodiments where the NSAID is a COX-2 inhibitor, the dosage can vary from about 100 ng/kg of body weight to 100 mg/kg of body weight per day and preferably will be from 1 mg/kg to 10 mg/kg per day.

It will be understood, however, that the specific dose level for any particular subject will depend upon a variety of factors including the activity of the specific compound employed, the half-life of the compound, the age, body weight, general health, sex, diet, time of administration, route of administration, rate of excretion, drug combination and the severity of the particular auto-immune disease or the severity of the need for immunosuppressive therapy. Dosage levels of immunosuppressant agents, for example cyclosporine can be on the order from about 0.05 mg to about 50 mg per kilogram of body weight per day are useful in the compositions and methods as disclosed herein (from about 2.5 mg to about 2.5 g per patient per day)). In some embodiments, a immunosuppressant, such as cyclosporine for use in the methods and compositions as disclosed herein can exceed 50 mg per kilogram of body weight per day (mg/kg/day) up to, or above the maximum tolerated dose (MTD) for the immunosuppressant, such as cyclosporine in humans.

The amount of NSAID and immunosuppressive agent can be combined with the carrier materials to produce a single dosage form will vary depending upon the subject host treated and the particular, mode of administration. For example, a formulation intended for the oral administration of humans may contain from 2.5 mg/kg to 2.5 g/kg of body weight of immunosuppressive agent and 100 ng/kg of body weight to 100 mg/kg of body weight per day of a NSAID with an appropriate and convenient amount of carrier material which may vary from about 5 to about 95 percent of the total composition. Dosage unit forms will generally contain between from about 5 mg to about 500 mg/kg per day body weight of immunosuppressive agent and 1 mg/kg to 10 mg/kg per day body weight of a NSAID.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

In some embodiments, the NSAIDs such as COX-2 selective inhibitor and immunosuppressive agents such as cyclosporine as disclosed herein can be provided in a therapeutic composition so that the preferred amounts of the NSAID such as COX-2 inhibitor and immunosuppressant such as cyclosporine are supplied by a single dosage, for example a single capsule enabling the NSAID and immunosuppressive agent to be administered to a subject at about the same time.

In some embodiments of the invention, a NSAID such as COX-2 inhibitor and an immunosuppressive agent such as cyclosporine, or an immunosuppressive agents analog, hydrolysis product, metabolite or precursor thereof can be administered substantially simultaneously, meaning that both agents can be provided in a single dosage, for example by mixing the agents and incorporating the mixture into a single capsule or within a short time of each other. In alternative embodiments, NSAIDs such as COX-2 inhibitor(s) and immunosuppressive agents such as cyclosporine can be administered substantially simultaneously by administration in separate dosages within a short time period, for example within one hour or less, 45 minutes or less, 30 minutes or less, 15 minutes or less, 10 minutes or less, 5 minutes or less and all time periods in between. Alternatively, a NSAID such as COX-2 inhibitor(s) and immunosuppressive agents such as cyclosporine can be administered sequentially, meaning that separate dosages, and possibly even separate dosage forms of NSAIDS such as COX-2 inhibitor(s) and immunosuppressive agents such as cyclosporine can be administered at separate times, for example on a staggered schedule but with equal frequency of administration of the a NSAID such as COX-2 inhibitor(s) and immunosuppressive agents such as cyclosporine. Of course, it is also possible that NSAIDS such as COX-2 inhibitor(s) can be administered either more or less frequently than immunosuppressive agents such as cyclosporine. Different agents have different half lives, thus one can stagger schedules and still maintain both agents being effective in an individual. In any case, it is preferable that, among successive time periods of a sufficient length, for example one day, the weight ratio of NSAIDS such as COX-2 inhibitor(s) administered to the weight ratio of immunosuppressive agents such as cyclosporine administered remains constant.

In some embodiments, the NSAID such as a COX-2 inhibitor and an immunosuppressive agent such as cyclosporine can be administered sequentially, for example the two agents can be administered within about 1 hour or more of each other, as long as they are both biologically active within the same time period. For example, the time between administration of the NSAID and the immunosuppressive agent can vary depending on the half life of the agent. For example a longer time period can occur between administration of the NSAID and the immunosuppressive if both agents have long half lives, as compared to a shorter time period between administration of the NSAID and the immunosuppressive if both agents have short half lives. Alternatively, the first agent administered for example a NSAID such as a COX-2 inhibitor can be administered in a time-release capsule to release the biologically active compound at a certain period after administration, or the NSAID such as a COX-2 inhibitor can be administered as a pro-drug that takes a certain time period to be metabolized to become the biologically active compound, and where in both situations the NSAID becomes biologically active at a time period which coincides with administration and biological activity of the immunosuppressive agent such as cyclosporine.

In alternative embodiments, a NSAID such as COX-2 inhibitor(s) and immunosuppressive agents such as cyclosporine can be administered sequentially, for example, one can administer a NSAID to a subject followed by an immunosuppressive agent, then a NSAID, and so forth, so that the subject is administered, in an alternating regimen, doses of a NSAID and such as COX-2 inhibitor(s) followed by doses of immunosuppressive agents such as cyclosporine.

In alternative embodiments, a subject is administered one agent continuously and administered the other agent in repeated doses. By way of an example but not as a limitation, a subject can be continuously administered an immunosuppressive agent by any suitable means such as catheterization or by pump administration and administered a NSAID at regular intervals, for example but not limited to daily, twice a day, twice a week, monthly etc by any suitable means known by persons of ordinary skill in the art and disclosed herein to keep the agents active in an individual.

In alternative embodiments, a subject is administered NSAIDS and immunosuppressive agents by pulse chase schedules. For example, a subject is administered one agent, such as a NSAID for a brief period of time (the pulse) and then a subject is administered the other agent, such as an immunosuppressive agent for a longer period (the chase). In such embodiments, a subject can be administered varying amounts of each agent for each pulse-chase administration regimen.

In some embodiments, a subject is administered varying amounts of each agent, for example varying amounts of a NSAID such as a COX-2 inhibitor and varying amounts of the immunosuppressive agent.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “biologically active” refers to a compound that is capable of eliciting a biological response in a tissue, system, animal or human.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included for use of the composition and method(s) as disclosed herein are the isomeric forms and tautomers and the pharmaceutically-acceptable salts of NSAIDS such as cyclooxygenase-2 selective inhibitors. Isomers of COX-2 inhibitors include their diastereomers, enantiomers, and racemates as well as their structural to isomers and are disclosed herein. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, α-hydroxybutyric, galactaric, and galacturonic acids.

Suitable pharmaceutically-acceptable base addition salts of compounds can be used in the compositions and methods as disclosed herein, such as for example, metallic ion salts and organic ion salts. More preferred metallic ion salts include, but are not limited to, appropriate alkali metal (group Ia) salts, alkaline earth metal (group Ia) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention. Pharmaceutically acceptable esters include, but are not limited to, the alkyl esters of the COX-2 inhibitors.

The terms “treating” or “to treat” means to alleviate a symptom or a malignancy or neoplasm or eliminate the causation either on a temporary or permanent basis, or to prevent or slow the appearance of a symptom. The term “treatment” includes alleviation, elimination of causation of, or prevention of, undesirable symptoms associated with immunosuppressive therapy, for example the prevention of a malignancy, tumor growth or neoplasia disorder. Besides being useful for human treatment, the compositions and methods as disclosed herein are useful for any subject in need of immunosuppressive therapy, for example these combinations are also useful for treatment of mammals, including horses, dogs, cats, rats, mice, sheep, pigs, etc.

The term “subject” for purposes of treatment includes any human or animal subject who is in need of immunosuppressive therapy. A subject is typically a human subject.

For methods of prevention of a malignancy or tumor growth in a subject in need thereof; for example a subject in need of immunosuppression can be any subject, for example any human or animal subject, and preferably is a subject that is in need of immunosuppressive therapy. A subject can be a human subject who is a recipient of an organ transplant, or is to undergo an organ transplant, or a subject who has an auto-immune disease. A subject can also be at risk for an auto-immune disease. In some embodiments, a subject can also be at risk of a neoplasia due to genetic predisposition, lifestyle, diet, exposure to disorder-causing agents, exposure to pathogenic agents and the like.

In connection with the inventive method, the compositions and methods as disclosed herein comprising NSAIDS, such as COX-2 inhibitors and immunosuppressive agents such as cyclosporine, immunosuppressive agent analogs, hydrolysis products, metabolites or precursors thereof can be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition can be at or near body temperature.

The phrase “administration” in defining the use of both NSAIDS such as cyclooxygenase-2 inhibitor agent and immunosuppressive agents such as cyclosporine is intended to encompass administration of each agent in a manner and in a regimen that will provide beneficial effects of the drug combination therapy, and is intended to encompass co-administration of more than 1, or more than 2 COX-2 inhibitors in a substantially simultaneous manner with one or more, or 2 or more immunosuppressive agents such as cyclosporine, or immunosuppressive agent analogs, hydrolysis products, metabolites or precursors thereof in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect or a therapeutically effective dose of the COX-2 inhibitor and a therapeutically effective dose of the immunosuppressive agents such as cyclosporine.

The phrases “therapeutically-effective” and “effective for the treatment, prevention, or inhibition”, are intended to qualify the amount of each NSAID such as a COX-2 agent and immunosuppressive agents such as cyclosporine for use in the immunosuppressive therapy which will achieve the goal of reduction of the incidence or severity and/or frequency of incidence of a tumor growth, malignancy or neoplasia associated with immunosuppression.

In some embodiments, the compositions and methods as disclosed herein comprising NSAID such as COX-2 inhibitors and immunosuppressive agents such as cyclosporine can be administered orally, for example, as tablets, coated tablets, dragees, troches, lozenges, gums, aqueous or oily suspensions, dispersible powders or granules, emulsions, hard or soft capsules, or syrups or elixirs. Compositions intended for oral use can be prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients can be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets can be uncoated or they can be coated by known techniques to delay disintegration and adsorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents can be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions can also contain one or more preservatives, for example, ethyl or n-propyl p-hydroxybenzoate, one or more coloring agents, one or more flavoring agents, or one or more sweetening agents, such as sucrose or saccharin.

Oily suspensions can be formulated by suspending the active ingredients in an omega-3 fatty acid, a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents, such as those set forth above, and flavoring agents can be added to provide a palatable oral preparation. These compositions can be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, can also be present.

Syrups and elixirs containing the novel combination can be formulated with sweetening agents, for example glycerol, sorbitol or sucrose. Such formulations may also contain demulcent, preservative and flavoring and coloring agents.

A pharmaceutical composition comprising NSAIDS such as COX-2 inhibitors and immunosuppressive agents such as cyclosporine as disclosed herein can also be administered parenterally, either subcutaneously, or intravenously, or intramuscularly, or intrasternally, or by infusion techniques, in the form of sterile injectable aqueous or olagenous suspensions. Such suspensions can be formulated according to the known art using those suitable dispersing of wetting agents and suspending agents which have been mentioned above, or other acceptable agents. The sterile injectable preparation can also be a sterile injectable solution or suspension in a non-toxic parenterally-acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, n-3 polyunsaturated fatty acids may find use in the preparation of injectables.

In some embodiments, composition as disclosed herein comprising NSAIDS such as COX-2 inhibitors and immunosuppressive agents such as cyclosporine can be formulated for targeted delivery to the colon. Formulation of the compositions comprising NSAIDS and immunosuppressive agents as disclosed herein for targeted delivery to the colon can be performed according the methods as disclosed in European Patent Applications EP825854 and EP827389 which are incorporated herein in their entirety by reference. Other examples of dosages and formulations for targeted delivery to the colon are disclosed in U.S. Pat. No. 5,171,580, which is incorporated herein by reference, teaches a preparation for delivery in the large intestine and especially the colon, comprising an active containing core coated with three protection layers of coatings having different solubilities. The inner layer is Eudragit® S, with a coating thickness of about 40-120 microns, the intermediate coating layer is a swellable polymer with a coating thickness of about 40-120 microns, and the outer layer is cellulose acetate phthalate, hydroxypropyl methyl cellulose phthalate, polyvinyl acetate phthalate, hydroxyethyl cellulose phthalate, cellulose acetate tetrahydrophthalate, or Eudragit® L. U.S. Pat. No. 4,910,021 which is incorporated herein by reference, teaches a targeted delivery system wherein the composition comprises a hard or soft gelatin capsule containing an active ingredient such as insulin and an absorption promoter. The capsule is coated with a film forming composition being sufficiently soluble at a pH above 7 as to be capable of permitting the erosion or dissolution of said capsule. The film forming composition is preferably a mixture of Eudragit® L, Eudragit® RS, and Eudragit® S at specific ratios to provide solubility above a pH of 7. U.S. Pat. No. 4,432,966 which is incorporated herein by reference, teaches a compressed tablet with an active agent, coated with a first coating layer comprising a mixture of microcrystalline cellulose and lower alkyl ether of a cellulose film-forming organic polymer such as ethyl cellulose, and a second coating layer selected from cellulose acetylphthalate, hydroxypropyl methylcellulose phthalate, benzophenyl salicylate, cellulose acetosuccinate, copolymers of styrene and of maleic acid, formulated gelatin, salol, keratin, stearic acid, myristic acid, gluten, acrylic and methacrylic resins, and copolymers of maleic acid and phthalic acid derivatives. EP-A-572,942 and EP-A-621,032 which are incorporated herein by reference, also describe colon specific dosage units with multiple polymer coatings. Each uses a pH independent inner layer and an outer coating like that of the inner layer of the present invention.

A pharmaceutical composition comprising NSAIDS such as COX-2 inhibitors and immunosuppressive agents such as cyclosporine as disclosed herein can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The compositions and methods as disclosed herein comprising NSAIDS such as COX-2 inhibitor(s) and immunosuppressive agents such as cyclosporine can also be administered topically, in the form of patches, creams, ointments, jellies, collyriums, solutions or suspensions. Of course, the compositions of the present invention can be administered by routes of administration other than topical administration. Also, as mentioned above, the COX-2 inhibitor(s) and immunosuppressive agents such as cyclosporine can be administered separately, with each agent administered by any of the above mentioned administration routes. As a non-limiting example, the compositions and methods as disclosed herein can be administered orally in any or the above mentioned forms (e.g. in capsule form) while the immunosuppressive agents such as cyclosporine is administered topically (e.g. as a cream).

Daily dosages can vary within wide limits and will be adjusted to the subject requirements in each particular case. In general, for administration to adults, an appropriate daily dosage has been described above, although the limits that were identified as being preferred can be exceeded if necessary. The daily dosage can be administered as a single dosage or in divided dosages. Various delivery systems include capsules, tablets, and gelatin capsules, for example.

Any suitable route and any combination of routes of administration can be employed for providing a subject with an effective dosage of a combined therapy of the present invention. For example, oral, rectal, transdermal, parenteral (subcutaneous, intramuscular, intravenous), intrathecal, and like forms of administration can be employed. Dosage forms include tablets, troches, dispersions, suspensions, solutions, capsules, patches, and the like.

The composition of the present invention is administered and dosed in accordance with good medical practice, taking into account the clinical condition of the individual subject, the site and method of administration, scheduling of administration, subject age, sex, body weight and other factors known to medical practitioners.

A pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the composition of the present invention can be administered in various ways. It should be noted that it can be administered as the compound or as pharmaceutically acceptable salt thereof and can be administered alone or as an active ingredient in combination with pharmaceutically acceptable carriers, diluents, adjuvants and vehicles. The composition can be administered orally, subcutaneously or parenterally including intravenous, intraarterial, intramuscular, intraperitoneally, and intranasal administration as well as intrathecal and infusion techniques. Implants of the composition are also useful. A subject being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

When administering the composition of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, potyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various other ingredients, as desired.

A pharmacological formulation of the present invention can be administered to a subject in an injectable formulation containing any compatible carrier, such as various vehicle, adjuvants, additives, and diluents; or the composition utilized in the present invention can be administered parenterally to a subject in the form of slow-release subcutaneous implants or targeted delivery systems such as monoclonal antibodies, vectored delivery, iontophoretic, polymer matrices, liposomes, microspheres and nanospheres. Examples of delivery systems useful in the present invention include those disclosed in U.S. Pat. Nos. 5,225,182; 5,169,383; 5,167,616; 4,959,217; 4,925,678; 4,487,603; 4,486,194; 4,447,233; 4,447,224; 4,439,196; and 4,475,196, which are incorporated herein by reference. Many other such implants, delivery systems, and modules are well known to those skilled in the art.

A pharmacological formulation of the composition utilized in the present invention can be administered orally to the subject. Conventional methods such as administering the composition in tablets, suspensions, solutions, emulsions, capsules, powders, syrups and the like are usable. Known techniques that deliver the composition orally or intravenously and retain the biological activity are preferred.

In another embodiment, the combination therapy of immunosuppressive agents such as cyclosporine and a COX-2 selective inhibitor can be administered alone or in conjunction with a standard tumor therapy, such as chemotherapy, immunotherapy, surgery, hormone therapy or radiation therapy or surgery.

While not wishing to be bound by any theory, the effect of the administration of a pharmaceutical compound comprising immunosuppressive agents such as cyclosporine and a NSAID, for example a COX-2 selective inhibitor as disclosed herein to a subject in need of immunosuppression, is useful to inhibit a malignancy or neoplasia or prevent the occurrence or increase of tumor growth. In some embodiments, the pharmaceutical compositions and methods as disclosed herein are administered in conjunction with the standard antitumor therapy and, in addition, can be administered on a continuing basis after the standard antitumor therapy. Chemotherapy or radiation therapy can then be repeated along with the continuation of the administration of the compositions and methods as disclosed herein comprising immunosuppressive agents such as cyclosporine and a COX-2 selective inhibitor.

In some embodiments, the compositions and methods as disclosed herein can also be used in combination with existing therapeutic agents for the treatment of cancer. Suitable agents to be used in combination include, but are not limited: (i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas), antimetabolites (for example antifolates such as fluoropyrimidines like 5-fluorouracil and tegafur, raltitrexed, methotrexate, cytosine arabinoside, hydroxyurea, gemcitabine and paclitaxel (Taxol[R]), antitumour antibiotics (for example anthracyclines like adriamycin, bleomycin, doxorubicin, daunomycin, epirubicin, idarubicin, mitomycin-C, dactinomycin and mithramycin), antimitotic agents (for example vinca alkaloids like vincristine, vinblastine, vindesine and vinorelbine and taxoids like taxol and taxotere), and topoisomerase inhibitors (for example epipodophyllotoxins like etoposide and teniposide, amsacrine, topotecan and camptothecin), (ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5 α-reductase such as finasteride, (iii) Agents which inhibit cancer cell invasion (for example metalloproteinase inhibitors like marimastat and inhibitors of urokinase plasminogen activator receptor function), (iv) inhibitors of growth factor function, for example such inhibitors include growth factor antibodies, growth factor receptor antibodies (for example the anti-erbb2 antibody trastuzumab [Herceptin] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI-1033)), for example inhibitors of the platelet-derived growth factor family and for example inhibitors of the hepatocyte growth factor family, (v) antiangiogenic agents such as those which inhibit the effects of vascular endothelial growth factor, (for example the anti-vascular endothelial cell growth factor antibody bevacizumab [Avastin], compounds such as those disclosed in International Patent Applications WO 97/22596, WO 97/30035, WO 97/32856 and WO 98/13354, which are incorporated herein by reference) and compounds that work by other mechanisms (for example linomide, inhibitors of integrin v³ function and angiostatin), (vi) vascular damaging agents such as Combretastatin A4 and compounds disclosed in International Patent Applications WO 99/02166, WO00/40529, WO 00/41669, WO01/92224, WO02/04434 and WO02/08213, which are incorporated herein by reference, (vii) antisense therapies, for example those which are directed to the targets listed above, such as ISIS 2503, an anti-ras antisense, (viii) gene therapy approaches, including for example approaches to replace aberrant genes such as aberrant p53 or aberrant BRCA 1 or BRCA2, GDEPT (gene-directed enzyme pro-drug therapy) approaches such as those using cytosine deaminase, thymidine kinase or a bacterial nitroreductase enzyme and approaches to increase subject tolerance to chemotherapy or radiotherapy such as multi-drug resistance gene therapy, and (ix) immunotherapy approaches, including for example ex-vivo and in-vivo approaches to increase the immunogenicity of subject tumor cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumor cell lines and approaches using anti-idiotypic antibodies.

In view of the above, it will be seen that the several advantages of the invention are achieved and other advantageous results obtained. As various changes could be made in the above methods and compositions without departing from the scope of the invention, it is intended that all matter contained in this application shall be interpreted as illustrative and not in a limiting sense.

Other embodiments within the scope of the embodiments herein will be apparent to one skilled in the art from consideration of the specification or practice of the invention as disclosed herein. It is intended that the specification be considered to be exemplary only, with the scope and spirit of the invention being indicated by the embodiments.

The discussion of the references herein is intended merely to summarize the assertions made by their authors and no admission is made that any reference constitutes prior art. Applicants reserve the right to challenge the accuracy and pertinency of the cited references.

### EXAMPLES

### Materials and Methods

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989, 2002) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory 385 Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (tn-cell) PCR in combination with Flow Cytometry can be used for detection of cells containing specific DNA and mRNA sequences (Testoni et al, 1996, Blood 87:3822.)

### Example 1

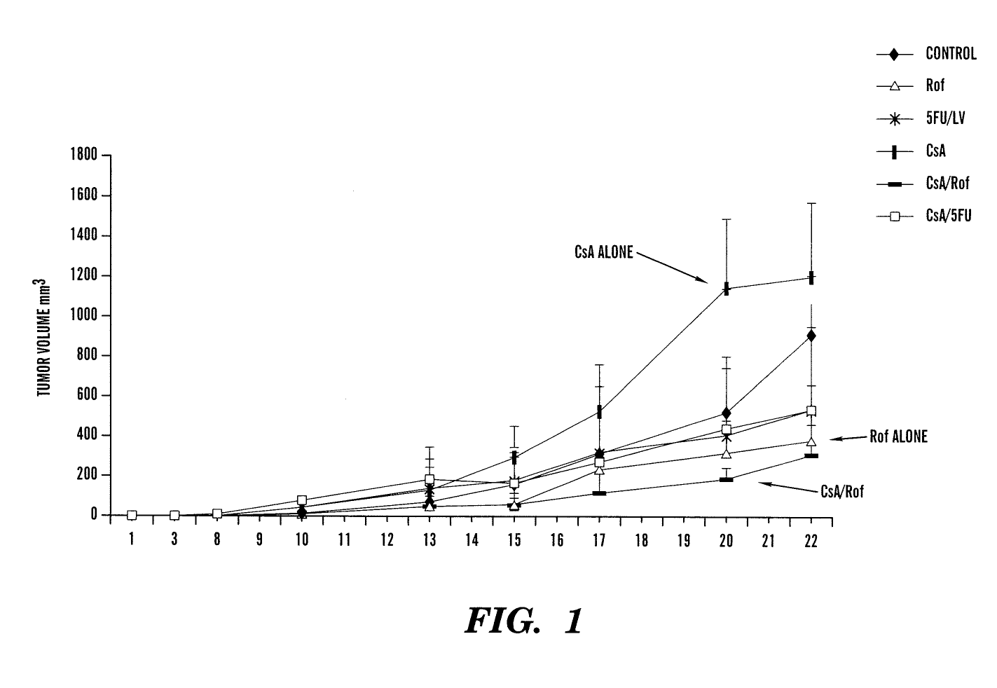

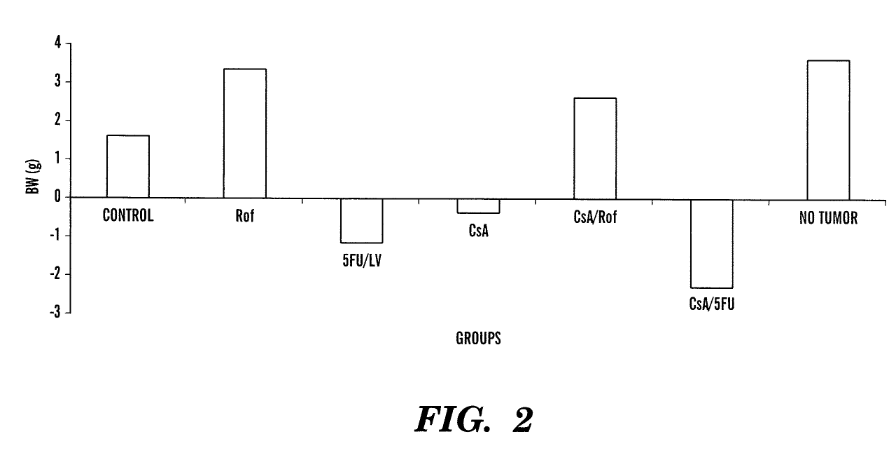

BALB/c mice were injected subcutaneously with MC-26 colorectal cancer cells and were then randomized to receive either no treatment (control), cyclosporine alone, rofecoxib-containing chow (equivalent to a human dose of 25 mg per day), a combination of rofecoxib and cyclosporine, 5-fluorocracil+leucovorin (5-FU/LV; “standard” therapy), or a combination of 5-FU/LV and cyclosporine for 22 days. A second control group included mice that were not injected with tumor cells and received no treatment. Whereas tumors grew significantly in control mice and in mice receiving cyclosporine only, tumors in mice receiving rofecoxib, and to a greater extent, in those receiving both rofecoxib and cyclosporine were significantly smaller than tumors in all other groups of mice (FIG. 1). Moreover, only mice receiving rofecoxib with or without cyclosporine continued to grow and gained weight similarly to the control group of mice that were not injected with tumor cells and received no therapy (FIG. 2). Thus, the combination of an NSAID and immunosuppressive agent effectively treats colorectal cancer with minimal toxicity.

### Example 2

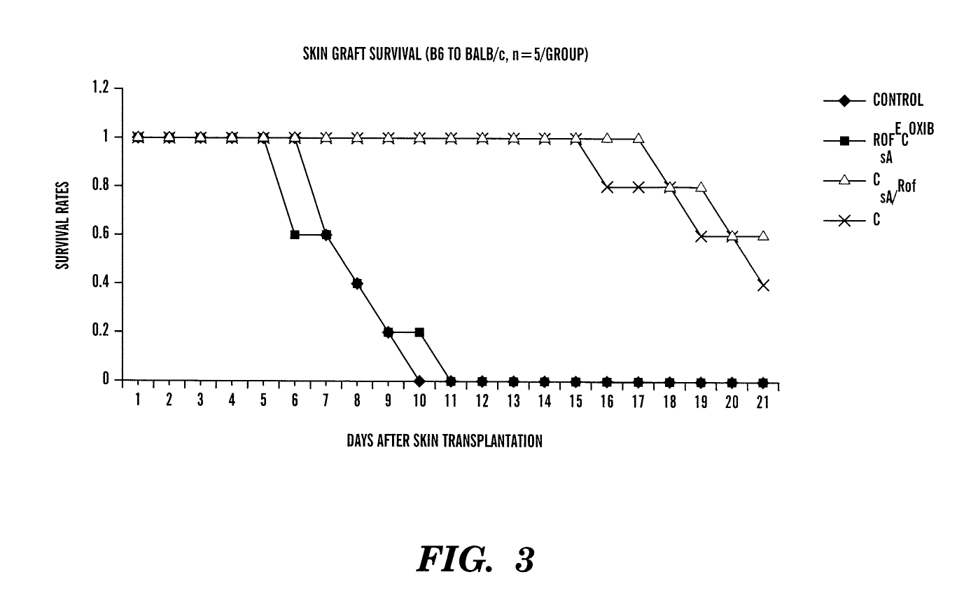

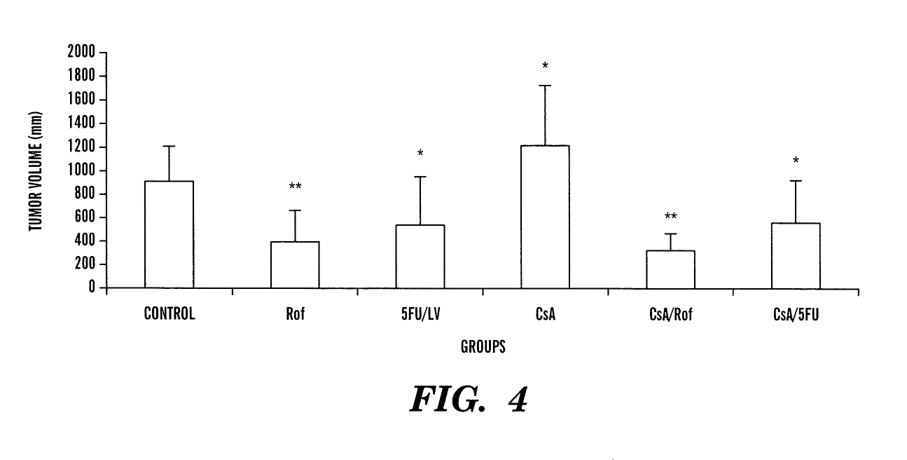

Skin from donor B6 black mice was transplanted to recipient white BALB/c mice. Animals were then randomized to receive either no treatment (control), cyclosporine (20 mg/kg) alone, rofecoxib-containing chow (equivalent to a human dose of 25 mg per day), or a combination of rofecoxib and cyclosporine for 22 days. Control recipient mice and recipient mice receiving rofecoxib alone rejected the skin graft, whereas mice receiving cyclosporine or cyclosporine plus rofecoxib did not reject the graft (FIG. 3). To determine the effects of these agents on secondary tumor development, BALB/c mice were also injected subcutaneously with MC-26 colorectal cancer cells and were treated for 22 days with the above regimens. A second control group included mice that were not injected with tumor cells and received no treatment. Whereas tumors grew significantly in control mice and in mice receiving cyclosporine only, tumors in mice receiving rofecoxib, and to a greater extent, in those receiving both rofecoxib and cyclosporine were significantly smaller (FIG. 4). Moreover, mice receiving rofecoxib with or without cyclosporine continued to grow and gained weight similarly to the control group of mice that were not injected with tumor cells and received no therapy. Thus, the inclusion of an NSAID to a regimen containing the immunosuppressive agent cyclosporine maintained the capacity to achieve adequate immunosuppression to prevent skin graft rejection while preventing tumor growth.

### Example 3

The inventors further demonstrated NSAIDs attenuated CRC tumor growth in the immunosuppressed transplant recipient, by assessing the effects of selective COX-2 inhibition and immunosuppression on CRC tumor progression and on skin graft survival in animals receiving these agents. The inventors subcutaneously injected 2×10⁴ MC-26 cells (mouse colorectal adenocarcinoma cell line) into the flanks of 6-8 week old BALB/c mice. Tumor volume was measured at regular intervals during a period of 22 days. Experimental groups included: (1) standard chow, no cyclosporine, (2) standard chow, +cyclosporine (20 mg/kg), (3) rofecoxib chow (0.01%), no cyclosporine, (4) rofecoxib chow+cyclosporine. Skin xenografts from donor black B6 mice were simultaneously transplanted to the backs of the mice. The inventors discovered tumor volume (TV) in BALB/c mice was markedly reduced in the group receiving rofecoxib compared with control chow (394.75±273.37 vs. 920.81±511.10 mm³). In contrast, cyclosporine enhanced tumor growth compared with control (1217.42±725.25 vs. 920.81±511.10 mm³). When rofecoxib chow was given to animals treated with cyclosporine, the inventors discovered tumor volume was decreased by 75% compared to the group receiving cyclosporine alone (317.13±149.97 vs. 1217.42±725.25 mm³). The inventors discovered the skin graft had failed by day 11 in control mice and in those receiving rofecoxib alone, whereas mice receiving cyclosporine did prolong graft survival, and COX-2 inhibition had no effect on graft survival. At day 15, skin graft survival was 100% in the groups receiving cyclosporine alone and the combination of cyclosporine and rofecoxib compared with 0% in control group and the group receiving rofecoxib alone. Accordingly, the inventors have discovered that cyclosporine alone enhanced tumor progression, whereas the addition of a COX-2 inhibitor attenuated tumor growth by 75%, while having no significant impact on skin graft survival. These results demonstrate that a combination of NSAIDs and immunosuppressive agents is an effective treatment for CRC and other malignancies. Accordingly, the inventors have discovered a method of using NSAIDs in combination with immunosuppressive agents such as cyclosporin to attenuate tumor growth for administration to subjects in need of immunosuppressive therapy such as transplant recipient patients, or subjects who have a higher incidence and mortality from CRC and other malignancies.

### REFERENCES

Throughout this application, various publications, including United States patents, are referenced by author and year and patents by number. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

1. A method of treating a malignancy or neoplasia disorder in a subject at risk thereof, comprising administering to the subject a cyclooxygenase-2 inhibitor or an isomer or pharmaceutically acceptable salt, ester or prodrug thereof in a first amount and an immunosuppressive agent in a second amount, wherein the first amount together with the second amount comprises a therapeutically effective amount for the treatment and/or prevention of a malignancy or neoplasia disorder in the subject.

2. A method of treating a subject receiving an immunosuppressive agent comprising administering an effective amount of a cyclooxygenase-2 inhibitor or an isomer, pharmaceutically acceptable salt, ester or prodrug thereof for an extended period of at least one month.

3. The method of claims 1 or 2, wherein the cyclooxygenase-2 inhibitor or isomer, pharmaceutically acceptable salt, ester, or prodrug thereof and the cyclosporine are administered substantially simultaneously for a period of at least one month.

4. The method of claims 1 or 2, wherein the cyclooxygenase-2 inhibitor is selected from the group comprising celecoxib (B-18), valdecoxib (B-19), deracoxib (B-20), rofecoxib (B-21), paracoxib etoricoxib (MK-663; B-22), JTE-522 (B-23), lumiracoxib, meloxicam, etodolac or an isomer, a pharmaceutically acceptable salt, ester, or prodrug thereof.

5. The method of claims 1 or 2, wherein the immunosuppressive agent is selected from the group consisting of cyclosporin, cyclosporin A, FK506, rapamycin, leflunomide, deoxyspergualin, prednisone, azathioprine, mycophenolate mofetil, OKT3, ATAG, mizoribine, mycophenolic acid, azathioprine or tacrolimus or an analog, hydrolysis product, metabolite or precursor thereof.

6. The method of claims 1 or 2, wherein the immunosuppressive agent is cyclosporin or an analog, hydrolysis product, metabolite or precursor thereof.

7. The method of claims 1 or 2, wherein the neoplasia disorder is a tumor growth or a malignant growth.

8. The method of claim 7, wherein the malignant growth is a colorectal cancer.

9. The method of claim 7, wherein the malignant growth is a viral-related cancer.

10. The method of claim 9, wherein the viral-related cancer is cervical cancer, T-cell leukemia, lymphoma, and Kaposi's sarcoma.

11. The method of claims 1 or 2, wherein the subject has mutations in genes resulting in colonic and extracolonic malignancies.

12. The method of claim 11, wherein the genes are selected from the group consisting of APC gene, hMLS1, hMSH2, hMSH6.

13. A pharmaceutical composition comprising a non-steroidal anti-inflammatory drug (NSAID) or a pharmaceutically acceptable salt, ester or prodrug thereof in an effective amount and an immunosuppressive agent in an effective amount, wherein the first amount together with the second amount comprises a therapeutically effective amount for the treatment, prevention or inhibition of a malignancy, or neoplasia disorder in the subject.

14. The pharmaceutical composition of claim 13, wherein the NSAID comprises a cyclooxygenase-2 (COX-2) inhibitor or isomer, a pharmaceutically acceptable salt, ester or prodrug thereof in a first amount and the immunosuppressive agent comprises cyclosporine.

15. The pharmaceutical composition of claim 14, wherein the cyclooxygenase-2 inhibitor is selected from the group comprising celecoxib (B-18), valdecoxib (B-19), deracoxib (B-20), rofecoxib (B-21), etoricoxib (MK-663; B-22), JTE-522 (B-23), lumiracoxib, meloxicam, etodolac or an isomer, a pharmaceutically acceptable salt, ester, or prodrug thereof.

16. (canceled)

17. (canceled)

18. (canceled)

19. (canceled)
